(19) United States
(12) Patent Application Publication
(10) Pub. No.: US 2025/0204779 A1
(43) Pub. Date: Jun. 26, 2025

(54) SYSTEMS AND METHODS FOR MULTISPECTRAL AND MOSAIC IMAGING

(71) Applicant: ACTIV Surgical, Inc., Boston, MA (US)

(72) Inventors: Mikael Marois, Boston, MA (US); John Oberlin, Boston, MA (US)

(21) Appl. No.: 18/851,536

(22) PCT Filed: Mar. 28, 2023
(86) PCT No.: PCT/US2023/016605

§ 371 Date: Sep. 26, 2024

Related U.S. Application Data
(60) Provisional application No. 63/325,083, filed on Mar. 29, 2022.

(51) Int. Cl. A61B 5/00

(52) U.S. Cl. CPC A61B 5/0035; A61B 5/0071; A61B 5/0077; A61B 2562/0233; A61B 2562/066; A61B 2576/00

(57) ABSTRACT

The present disclosure provides a system for medical imaging. The system may comprise one or more imaging sensors for imaging a surgical scene. Each of the one or more imaging sensors may comprise a plurality of pixels. At least one pixel of the plurality of pixels may comprise a plurality of sub-pixels sensitive to different bands or wavelengths of light. The system may further comprise a processing unit operatively coupled to the one or more imaging sensors. The processing unit may be configured to perform a quantitative analysis of one or more features or fiducials that are detectable within the surgical scene based on one or more light signals obtained or registered using the plurality of sub-pixels.

### CROSS-REFERENCE

This application claims the benefit of U.S. Provisional Application No. 63/325,083, filed Mar. 29, 2022, which application is incorporated herein by reference in its entirety.

### BACKGROUND

Medical imaging data may be used to aid in the diagnosis and/or treatment of different medical conditions, and the performance of various medical or surgical procedures. Such medical imaging data may be associated with various anatomical, physiological, or morphological features within a surgical scene.

### SUMMARY

The systems and methods disclosed herein may be used to generate accurate and useful multispectral, hyperspectral, and/or mosaic imaging datasets that can be leveraged by medical or surgical operators for a variety of different applications or surgical procedures. The systems and methods of the present disclosure can be used to provide a medical or surgical operator with additional visual information of a surgical scene, including, for example, real time multispectral, hyperspectral, and/or mosaic image overlays to enhance a medical operator's ability to perform a live surgical procedure in an optimal manner. In some cases, the multispectral, hyperspectral, and/or mosaic images generated using the systems and methods of the present disclosure may also be used to improve the precision, flexibility, and control of autonomous and/or semiautonomous robotic surgical systems.

The systems and methods of the present disclosure may be implemented for medical imaging of a surgical scene using one or more sensors capable of imaging in a variety of different imaging modalities. The medical images obtained or generated using the presently disclosed systems and methods may comprise, for example, fluorescence images (including autofluorescence images based on tissue autofluorescence characteristics and/or fluorescence images generated based on fluorescent dyes or markers), RGB images, depth maps, time of flight (TOF) images, laser speckle contrast images, hyperspectral images, multispectral images, mosaic images, or laser doppler images. The medical images may also comprise, for example, fluorescence videos (including autofluorescence videos based on tissue autofluorescence characteristics and fluorescence videos generated based on fluorescent dyes or markers), time of flight (TOF) videos, RGB videos, dynamic depth maps, laser speckle contrast videos, hyperspectral videos, multispectral videos, mosaic videos or laser doppler videos. In some cases, the medical imagery may comprise one or more streams of imaging data comprising one or more medical images. The one or more streams of imaging data may comprise a series of medical images obtained successively or sequentially over a time period.

In some embodiments, the medical images may be processed to determine or detect one or more anatomical, physiological, or morphological processes or properties associated with the surgical scene or the subject undergoing a surgical procedure. As used herein, processing the medical images may comprise determining or classifying one or more features, patterns, or attributes of the medical images. In some embodiments, the medical images may be used to train or implement one or more medical algorithms or models for tissue tracking. In some embodiments, the systems and methods of the present disclosure may be used to augment various medical imagery with fluorescence information associated with a surgical scene.

In some embodiments, the one or more medical images may be used or processed to provide live guidance based on a detection of one or more tools, surgical phases, critical views, or one or more biological, anatomical, physiological, or morphological features in or near the surgical scene. In some embodiments, the one or more medical images may be used to enhance intra-operative decision making and provide supporting features (e.g., enhanced image processing capabilities or live data analytics) to assist a surgeon during a surgical procedure.

In some embodiments, the one or more medical images may be used to generate an overlay comprising (i) one or more RGB images or videos of the surgical scene and (ii) one or more additional images or videos of the surgical procedure, wherein the one or more additional images or videos comprise fluorescence data, laser speckle data, perfusion data, or depth information.

The multispectral, hyperspectral, and/or mosaic imaging systems and methods disclosed herein may provide several advantages over other conventional imaging systems. For example, the presently disclosed systems and methods may be used to obtain multispectral, hyperspectral, and/or mosaic images of a target region or one or more objects or features in the target region using a single sensor. The single sensor may comprise a plurality of pixels for imaging in different wavelengths or different ranges of wavelengths. Such a sensor can permit multi-wavelength imaging and/or imaging based on various different imaging modalities without the need for multiple sensors that are configured for imaging specifically in a single wavelength or a single range of wavelengths. By utilizing a sensor comprising multiple pixels that are capable of multi-wavelength imaging, multispectral, hyperspectral, and/or mosaic imaging can be performed using a simplified and compact system that is adaptable for imaging across multiple different imaging modalities.

The systems and methods of the present disclosure also allow for accurate and reliable quantitative analysis of signals transmitted or reflected from the target site using sensors that are individually capable of multispectral, hyperspectral, and/or mosaic imaging. Such quantitative analysis may provide additional information about the target site being imaged, including, for instance, an amount of fluorescent material present or an amount of blood present within or flowing through the target site. Such quantitative analysis may provide a doctor or a surgeon with a numerical basis for interpreting and understanding processes or features of interest in the target site.

In an aspect, the present disclosure provides a system. The system may comprise one or more imaging sensors configured to capture an image of a tissue, wherein each of the one or more imaging sensors comprises a plurality of pixels, wherein at least one pixel of the plurality of pixels comprises a first plurality of sub-pixels sensitive to a first band or wavelength of light and a second plurality of sub-pixels sensitive to a second band or wavelength of light, wherein the first band or wavelength of light is distinct from the second band or wavelength of light; and a processing unit operatively coupled to the one or more imaging sensors, wherein the processing unit is configured to perform a quantitative analysis of one or more features or fiducials that are detectable within the image of the tissue based on one or more light signals obtained or registered using each of the first plurality of sub-pixels and the second plurality of sub-pixels.

In some embodiments, the system further comprises: a first optical illumination in a third band or wavelength of light and a second optical illumination in a fourth band or wavelength of light, and, optionally, wherein the third band or wavelength of light and the fourth band or wavelength of light are distinct. In some embodiments, the first optical illumination is selected to generate data for the first plurality of sub-pixels sensitive to the first band or wavelength of light, and wherein the second optical illumination is selected to generate data for the second plurality of sub-pixels sensitive to the second band or wavelength of light.

In some embodiments, each of the first plurality of sub-pixels and the second plurality of sub-pixels are configured to generate image data comprising distinct image modalities, and, optionally, wherein a first image modality is a color image and a second image modality is fluorescence imaging or laser speckle imaging. In some embodiments, the processing unit is configured to collect the one or more light signals from each of the first plurality of sub-pixels and the second plurality of sub-pixels substantially in parallel. In some embodiments, the processing unit is configured to perform the quantitative analysis substantially in real time based on the one or more light signals collected substantially in parallel.

In some embodiments, the quantitative analysis comprises a quantification an amount of fluorescence emitted from the surgical scene or a concentration of a fluorescing material or substance. In some embodiments, the one or more features or fiducials comprise the fluorescing material or substance. In some embodiments, the one or more features or fiducials comprise fluorescence from fluorescein, methylene blue, indigo carmine, patent blue, Indocyanine Green, Protoporphyrin IX (PPIX) or riboflavin (B2) in the fluorescing material or substance.

In some embodiments, the processing unit is configured to determine the quantification using spectral fitting or absorption spectroscopy. In some embodiments, the quantitative analysis comprises an identification or classification of one or more tissue regions in the tissue based on the one or more light signals. In some embodiments, the quantitative analysis comprises a multispectral classification of one or more tissue regions in the tissue based on the one or more light signals. In some embodiments, the one or more light signals comprise a plurality of different wavelengths. In some embodiments, the quantitative analysis comprises a determination of the real-time blood oxygenation based on the one or more light signals. In some embodiments, the quantitative analysis comprises a quantitative speckle analysis based on the one or more light signals.

In some embodiments, the one or more light signals comprise fluorescent light emitted by a fluorescent material or tissue autofluorescence light. In some embodiments, the processing unit is configured to quantify an amount of fluorescence emitted from the tissue or an amount of fluorescent material present in the tissue based on a lighting condition of the image, wherein the lighting condition comprises an illumination bias, an illumination profile, or an illumination gradient of the image.

In some embodiments, the first band or wavelength of light and the second band or wavelength of light correspond to distinct bands or wavelengths of visible light, infrared light, or ultraviolet light. In some embodiments, the first band or wavelength of light is within the infrared, and wherein the second band or wavelength of light is in the visible or the ultraviolet. In some embodiments, the first band or wavelength of light is within the visible, and wherein the second band or wavelength of light is in the infrared or the ultraviolet. In some embodiments, the first band or wavelength of light is within the ultraviolet, and wherein the second band or wavelength of light is in the infrared or the visible. In some embodiments, the first band or wavelength of light and the second band or wavelength of light range from about 10 nanometers (nm) to about 1 centimeter (cm). In some embodiments, the system further comprises one or more band pass filters for filtering out one or more bands or wavelengths of light emitted, reflected, or received from the tissue.

In some embodiments, the processing unit is configured to generate one or more combined images of the tissue based on image data or image signals derived from each of the first plurality of sub-pixels and the second plurality of sub-pixels. In some embodiments, the processing unit is configured to generate a quantitative map of fluorescence in the tissue based on the image data or image signals. In some embodiments, the quantitative map of fluorescence indicates an amount or concentration of fluorescence material present in one or more regions of the tissue. In some embodiments, the processing unit is configured to perform a calibration that correlates an amount of fluorescent light detected by the one or more imaging sensors to the amount or concentration of fluorescent material present in the one or more regions.

In some embodiments, the processing unit is configured to (i) estimate an amount of blood in the tissue and (ii) determine an amount or a concentration of fluorophores or fluorescent material present in the tissue based on (a) the estimated amount of blood and (b) at least a subset of the one or more light signals. In some embodiments, the one or more light signals comprise light that is emitted, reflected, or received from the tissue. In some embodiments, the one or more light signals comprise light having a plurality of wavelengths suitable for visible light imaging, near infrared imaging, short-wave infrared imaging, mid-wave infrared imaging, or long-wave infrared imaging. In some embodiments, the plurality of pixels comprises greater than 10 sets of sub-pixels each comprising a distinct wavelength. In some embodiments, the plurality of pixels comprises greater than 100 sets of sub-pixels each comprising a distinct wavelength.

In another aspect, the present application provides a method of quantitative imaging using multispectral images. The method may comprise providing an image of a tissue, wherein the image comprises data from one or more imaging sensors, wherein each of the one or more imaging sensors comprises a plurality of pixels, wherein at least one pixel of the plurality of pixels comprises a first plurality of sub-pixels sensitive to a first band or wavelength of light and a second plurality of sub-pixels sensitive to a second band or wavelength of light, wherein the first band or wavelength of light is distinct from the second band or wavelength of light; and at a processing unit operatively coupled to the one or more imaging sensors, performing a quantitative analysis of one or more features or fiducials that are detectable within the image of the tissue based on one or more light signals obtained or registered using each of the first plurality of sub-pixels and the second plurality of sub-pixels.

In some embodiments, the image of the tissue comprises data from a first optical illumination in a third band or wavelength of light and a second optical illumination in a fourth band or wavelength of light. In some embodiments, the third band or wavelength of light and the fourth band or wavelength of light are distinct. In some embodiments, the first optical illumination is selected to generate data for the first plurality of sub-pixels sensitive to the first band or wavelength of light, and wherein the second optical illumination is selected to generate data for the second plurality of sub-pixels sensitive to the second band or wavelength of light.

In some embodiments, the method further comprises, at a processing unit, collecting the one or more light signals from each of the first plurality of sub-pixels and the second plurality of sub-pixels substantially in parallel. In some embodiments, the method further comprises, at a processing unit, performing the quantitative analysis substantially in real time based on the one or more light signals collected substantially in parallel. In some embodiments, the quantitative analysis comprises a quantification an amount of fluorescence emitted from the surgical scene or a concentration of a fluorescing material or substance. In some embodiments, the one or more features or fiducials comprise the fluorescing material or substance. In some embodiments, the one or more features or fiducials comprise fluorescence from fluorescein, methylene blue, indigo carmine, patent blue, Indocyanine Green, Protoporphyrin IX (PPIX) or riboflavin (B2) in the fluorescing material or substance. In some embodiments, the method further comprises, at a processing unit, determining the quantification using spectral fitting or absorption spectroscopy.

In some embodiments, the quantitative analysis comprises an identification or classification of one or more tissue regions in the tissue based on the one or more light signals. In some embodiments, the quantitative analysis comprises a multispectral classification of one or more tissue regions in the tissue based on the one or more light signals. In some embodiments, the one or more light signals comprise a plurality of different wavelengths. In some embodiments, the quantitative analysis comprises a determination of the real-time blood oxygenation based on the one or more light signals. In some embodiments, the quantitative analysis comprises a quantitative speckle analysis based on the one or more light signals. In some embodiments, the one or more light signals comprise fluorescent light emitted by a fluorescent material or tissue autofluorescence light.

In some embodiments, the method further comprises, at a processing unit, quantifying an amount of fluorescence emitted from the tissue or an amount of fluorescent material present in the tissue based on a lighting condition of the image, wherein the lighting condition comprises an illumination bias, an illumination profile, or an illumination gradient of the image. In some embodiments, the first band or wavelength of light and the second band or wavelength of light correspond to distinct bands or wavelengths of visible light, infrared light, or ultraviolet light. In some embodiments, the first band or wavelength of light is within the infrared, and wherein the second band or wavelength of light is in the visible or the ultraviolet. In some embodiments, the first band or wavelength of light is within the visible, and wherein the second band or wavelength of light is in the infrared or the ultraviolet. In some embodiments, the first band or wavelength of light is within the ultraviolet, and wherein the second band or wavelength of light is in the infrared or the visible. In some embodiments, the first band or wavelength of light and the second band or wavelength of light range from about 10 nanometers (nm) to about 1 centimeter (cm).

In some embodiments, the method further comprises, at a processing unit, generating one or more combined images of the tissue based on image data or image signals derived from each of the first plurality of sub-pixels and the second plurality of sub-pixels. In some embodiments, the method further comprises, at a processing unit, generating a quantitative map of fluorescence in the tissue based on the image data or image signals. In some embodiments, the quantitative map of fluorescence indicates an amount or concentration of fluorescence material present in one or more regions of the tissue. In some embodiments, the method further comprises, at a processing unit, performing a calibration that correlates an amount of fluorescent light detected by the one or more imaging sensors to the amount or concentration of fluorescent material present in the one or more regions. In some embodiments, the method further comprises, at a processing unit, (i) estimating an amount of blood in the tissue and (ii) determining an amount or a concentration of fluorophores or fluorescent material present in the tissue based on (a) the estimated amount of blood and (b) at least a subset of the one or more light signals.

In some embodiments, the one or more light signals comprise light that is emitted, reflected, or received from the tissue. In some embodiments, the one or more light signals comprise light having a plurality of wavelengths suitable for visible light imaging, near infrared imaging, short-wave infrared imaging, mid-wave infrared imaging, or long-wave infrared imaging. In some embodiments, the plurality of pixels comprises greater than 10 sets of sub-pixels each comprising a distinct wavelength. In some embodiments, the plurality of pixels comprises greater than 100 sets of sub-pixels each comprising a distinct wavelength. In some embodiments, each of the first plurality of sub-pixels and the second plurality of sub-pixels are configured to generate image data comprising distinct image modalities, and, optionally, wherein a first image modality is a color image and a second image modality is fluorescence imaging or laser speckle imaging. In some embodiments, the method further comprises, at a processing unit, generating image data comprising distinct image modalities from each of the first plurality of sub-pixels and the second plurality of sub-pixels, and, optionally, wherein a first image modality is a color image and a second image modality is fluorescence imaging or laser speckle imaging.

In another aspect, the present disclosure provides a method of quantitative imaging using multispectral images. The method may comprise providing the system of any aspect or embodiment.

In another aspect, the present disclosure provides a non-transitory computer readable medium with instructions stored thereon which when implemented by a processor are configured to perform the method of any aspect or embodiment.

In another aspect, the present disclosure provides a system. The system may comprise a processing unit comprising an image of a tissue, wherein the image comprises data from one or more imaging sensors, wherein each of the one or more imaging sensors comprises a plurality of pixels, wherein at least one pixel of the plurality of pixels comprises a first plurality of sub-pixels sensitive to a first band or wavelength of light and a second plurality of sub-pixels sensitive to a second band or wavelength of light, wherein the first band or wavelength of light is distinct from the second band or wavelength of light, and wherein the processing unit is configured to perform a quantitative analysis of one or more features or fiducials that are detectable within the image of the tissue based on one or more light signals obtained or registered using each of the first plurality of sub-pixels and the second plurality of sub-pixels.

In some embodiments, the system further comprises: a first optical illumination in a third band or wavelength of light and a second optical illumination in a fourth band or wavelength of light, and, optionally, wherein the third band or wavelength of light and the fourth band or wavelength of light are distinct. In some embodiments, the first optical illumination is selected to generate data for the first plurality of sub-pixels sensitive to the first band or wavelength of light, and wherein the second optical illumination is selected to generate data for the second plurality of sub-pixels sensitive to the second band or wavelength of light. In some embodiments, each of the first plurality of sub-pixels and the second plurality of sub-pixels are configured to generate image data comprising distinct image modalities, and, optionally, wherein a first image modality is a color image and a second image modality is fluorescence imaging or laser speckle imaging.

In some embodiments, the processing unit is configured to collect the one or more light signals from each of the first plurality of sub-pixels and the second plurality of sub-pixels substantially in parallel. In some embodiments, the processing unit is configured to perform the quantitative analysis substantially in real time based on the one or more light signals collected substantially in parallel. In some embodiments, the quantitative analysis comprises a quantification an amount of fluorescence emitted from the surgical scene or a concentration of a fluorescing material or substance. In some embodiments, the one or more features or fiducials comprise the fluorescing material or substance. In some embodiments, the one or more features or fiducials comprise fluorescence from fluorescein, methylene blue, indigo carmine, patent blue, Indocyanine Green, Protoporphyrin IX (PPIX) or riboflavin (B2) in the fluorescing material or substance. In some embodiments, the processing unit is configured to determine the quantification using spectral fitting or absorption spectroscopy.

In some embodiments, the quantitative analysis comprises an identification or classification of one or more tissue regions in the tissue based on the one or more light signals. In some embodiments, the quantitative analysis comprises a multispectral classification of one or more tissue regions in the tissue based on the one or more light signals. In some embodiments, the one or more light signals comprise a plurality of different wavelengths. In some embodiments, the quantitative analysis comprises a determination of the real-time blood oxygenation based on the one or more light signals. In some embodiments, the quantitative analysis comprises a quantitative speckle analysis based on the one or more light signals.

In some embodiments, the one or more light signals comprise fluorescent light emitted by a fluorescent material or tissue autofluorescence light. In some embodiments, the processing unit is configured to quantify an amount of fluorescence emitted from the tissue or an amount of fluorescent material present in the tissue based on a lighting condition of the image, wherein the lighting condition comprises an illumination bias, an illumination profile, or an illumination gradient of the image.

In some embodiments, the first band or wavelength of light and the second band or wavelength of light correspond to distinct bands or wavelengths of visible light, infrared light, or ultraviolet light. In some embodiments, the first band or wavelength of light is within the infrared, and wherein the second band or wavelength of light is in the visible or the ultraviolet. In some embodiments, the first band or wavelength of light is within the visible, and wherein the second band or wavelength of light is in the infrared or the ultraviolet. In some embodiments, the first band or wavelength of light is within the ultraviolet, and wherein the second band or wavelength of light is in the infrared or the visible. In some embodiments, the first band or wavelength of light and the second band or wavelength of light range from about 10 nanometers (nm) to about 1 centimeter (cm). In some embodiments, they system further comprises one or more band pass filters for filtering out one or more bands or wavelengths of light emitted, reflected, or received from the tissue.

In some embodiments, the processing unit is configured to generate one or more combined images of the tissue based on image data or image signals derived from each of the first plurality of sub-pixels and the second plurality of sub-pixels. In some embodiments, the processing unit is configured to generate a quantitative map of fluorescence in the tissue based on the image data or image signals. In some embodiments, the quantitative map of fluorescence indicates an amount or concentration of fluorescence material present in one or more regions of the tissue. In some embodiments, the processing unit is configured to perform a calibration that correlates an amount of fluorescent light detected by the one or more imaging sensors to the amount or concentration of fluorescent material present in the one or more regions. In some embodiments, the processing unit is configured to (i) estimate an amount of blood in the tissue and (ii) determine an amount or a concentration of fluorophores or fluorescent material present in the tissue based on (a) the estimated amount of blood and (b) at least a subset of the one or more light signals.

In some embodiments, the one or more light signals comprise light that is emitted, reflected, or received from the tissue. In some embodiments, the one or more light signals comprise light having a plurality of wavelengths suitable for visible light imaging, near infrared imaging, short-wave infrared imaging, mid-wave infrared imaging, or long-wave infrared imaging. In some embodiments, the plurality of pixels comprises greater than 10 sets of sub-pixels each comprising a distinct wavelength. In some embodiments, the plurality of pixels comprises greater than 100 sets of sub-pixels each comprising a distinct wavelength.

In another aspect, the present disclosure provides a system for medical imaging. The system may comprise one or more imaging sensors configured to image a surgical scene. Each of the one or more imaging sensors may comprise a plurality of pixels. At least one pixel of the plurality of pixels may comprise a plurality of sub-pixels sensitive to different bands or wavelengths of light. The system may also comprise a processing unit operatively coupled to the one or more imaging sensors. The processing unit may be configured to perform a quantitative analysis of one or more features or fiducials that are detectable within the surgical scene based on one or more light signals obtained or registered using the plurality of sub-pixels.

In some cases, the quantitative analysis comprises quantifying an amount of fluorescence emitted from the surgical scene or a concentration of a material or substance emitting the fluorescence. In some cases, the one or more features or fiducials comprise the material or substance emitting the fluorescence. In some cases, the one or more features or fiducials comprise Protoporphyrin IX (PPIX). In some cases, the quantitative analysis comprises identifying or classifying one or more tissue regions in the surgical scene based on the one or more light signals. In some cases, the quantitative analysis comprises multispectral classification of one or more tissue regions in the surgical scene based on the one or more light signals. In some cases, the one or more light signals comprise a plurality of different wavelengths.

In some cases, the quantitative analysis comprises determining real-time blood oxygenation based on the one or more light signals. In some cases, the quantitative analysis comprises quantitative ICG analysis and/or quantitative speckle analysis based on the one or more light signals. In some cases, the processing unit is configured to determine an amount or a concentration of fluorescent material present in the surgical scene using spectral fitting and/or absorption spectroscopy. In some cases, the one or more light signals comprise fluorescent light emitted by a fluorescent material or tissue autofluorescence light. In some cases, the processing unit is configured to quantify an amount of fluorescence emitted from the surgical scene or an amount of fluorescent material present in the surgical scene based on a lighting condition of the surgical scene, wherein the lighting condition comprises an illumination bias, an illumination profile, or an illumination gradient of the surgical scene.

In some cases, the plurality of pixels is configured to capture different spectral wavelengths of light. In some cases, the different spectral wavelengths of light correspond to at least two of visible light, infrared light, and ultraviolet light. In some cases, the different spectral wavelengths of light range from about 10 nanometers (nm) to about 1 centimeter (cm). In some cases, the system further comprises one or more band pass filters for filtering out one or more bands or wavelengths of light emitted, reflected, or received from the surgical scene. In some cases, the one or more light signals obtained using the plurality of sub-pixels comprise at least one of visible light, infrared light, and fluorescent light.

In some cases, the processing unit is configured to generate one or more images of the surgical scene based on image data or image signals derived from the one or more light signals. In some cases, the processing unit is configured to generate a quantitative map of fluorescence in the surgical scene based on the image data or image signals. In some cases, the quantitative map of fluorescence indicates an amount or concentration of fluorescence material present in one or more regions of the surgical scene. In some cases, the processing unit is configured to perform a calibration that correlates an amount of fluorescent light detected by the one or more imaging sensors to the amount or concentration of fluorescent material present in the one or more regions.

In some cases, the processing unit is configured to (i) estimate an amount of blood in the surgical scene and (ii) determine an amount or a concentration of fluorophores or fluorescent material present in the surgical scene based on (a) the estimated amount of blood and (b) at least a subset of the one or more light signals. In some cases, at least one pixel of the plurality of pixels is configured for fluorescence imaging, laser speckle imaging, RGB imaging, and/or depth imaging. In some cases, the one or more light signals comprise light that is emitted, reflected, or received from the surgical scene. In some cases, the one or more light signals comprise light having a plurality of wavelengths suitable for visible light imaging, near infrared imaging, short-wave infrared imaging, mid-wave infrared imaging, and/or long-wave infrared imaging.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates a system for multispectral, hyperspectral, and/or mosaic imaging, in accordance with some embodiments.

FIG. 2 schematically illustrates a system for multispectral, hyperspectral, and/or mosaic imaging that comprises one or more imaging sensors that can register light from one or more light sources, in accordance with some embodiments.

FIG. 3 schematically illustrates a system for multispectral, hyperspectral, and/or mosaic imaging comprising an imaging module, an image processing unit, a calibration module, and a display unit, in accordance with some embodiments.

FIG. 4 schematically illustrates ICG absorption and fluorescence characteristics that can be leveraged to perform the quantitative fluorescence applications described herein.

FIG. 5A and FIG. 5B show various plots of blood oxygenation based on imaging data obtained using a spectrometer and a hyperspectral imaging camera.

FIG. 6A and FIG. 6B schematically illustrate various examples of medical images obtained using one or more multispectral, hyperspectral, and/or mosaic imaging sensors.

FIG. 7 schematically illustrates an example of a processing unit configured to perform multispectral classification based on a baseline RGB image and additional imaging data derived from the light signals registered using a multispectral, hyperspectral, and/or mosaic imaging sensor.

FIG. 8 schematically illustrates a computer system that is programmed or otherwise configured to implement methods provided herein.

FIG. 9 schematically illustrates absorption and emission characteristics for PPIX.

FIG. 10A and FIG. 10B show an example of a surgical scene where PPIX has accumulated in one area of the tissue.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “real-time,” as used herein, generally refers to a simultaneous or substantially simultaneous occurrence of a first event or action with respect to an occurrence of a second event or action. A real-time action or event may be performed within a response time of less than one or more of the following: ten seconds, five seconds, one second, a tenth of a second, a hundredth of a second, a millisecond, or less relative to at least another event or action. A real-time action may be performed by one or more computer processors.

Whenever the term “at least,” “greater than” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Imaging

In one aspect, the present disclosure provides a system for medical imaging. The system may be configured for multispectral, hyperspectral, and/or mosaic imaging.

In some cases, multispectral imaging may comprise spectral imaging using a plurality of discrete wavelength bands. In some cases, a multispectral sensor comprises an imaging sensor comprising a plurality of pixels, wherein at least one pixel of the plurality of pixels comprises a first plurality of sub-pixels sensitive to a first band or wavelength of light and a second plurality of sub-pixels sensitive to a second band or wavelength of light. In some cases, the first band or wavelength of light is distinct from the second band or wavelength of light.

In some cases, hyperspectral imaging comprises imaging a plurality of spectral wavelength bands over a continuous spectral range. A hyperspectral imaging sensor may comprise a plurality of narrow wavelength bands. In some cases, hyperspectral imaging may comprise a type of multispectral imaging with a greater number of wavelength bands. In some cases, hyperspectral imaging may comprise capturing intensity information at each pixel coordinate across many wavelength bands other than the standard red, blue, and green (RBG) colors, thereby providing increased insight into tissue oxygenation and blood perfusion.

In some cases, mosaic imaging comprises imaging a plurality of spectral wavelength bands using a mosaic imaging sensor.

The absorption, reflection, and scattering of light incident on a biological material or a physiological feature may depend on the chemical properties of the material or feature as well as the imaging wavelength used, and images obtained from additional spectra as in hyperspectral imaging can include information on compositions, concentrations, or other properties or characteristics of a surgical scene that is difficult to visualize using standard RGB imaging or the human eye.

### Imaging—Imaging Sensors

In some embodiments, the multispectral, hyperspectral, and/or mosaic imaging may be performed using one or more imaging sensors. The imaging sensors may comprise an array of light-detecting elements, which may detect an intensity of light incident on the light-detecting elements. In some embodiments, the one or more imaging sensors may comprise a multispectral, hyperspectral, and/or mosaic imaging sensor. Multispectral, hyperspectral, and/or mosaic imaging sensors may offer advantages over traditional color sensors. Bands in color cameras may not be advantageous for computational measurements because absorption may not be flat across the bands. For example, the amplitude response for a red pixel may not be the same as a green pixel for the same light intensity. Multispectral, hyperspectral, and mosaic cameras may generate more computational accurate imaging without use of a spectrophotometer.

The multispectral sensor may be configured for imaging in a plurality of different wavelengths. The plurality of different wavelengths may lie in the visible light spectrum, the infrared light spectrum, the near infrared light spectrum, the short-wave infrared spectrum, the mid wave infrared spectrum, and/or the long wave infrared spectrum. In any of the embodiments described herein, the multispectral sensor may be configured for imaging in a plurality of different wavelength bands. The different spectral wavelengths may be registered at different pixels or sub-pixels of the imaging sensor, as described in greater detail below. In some cases, the pixels or sub-pixels of the imaging sensor may be capable of generating imaging data associated with multiple different wavelengths or spectral ranges.

The multispectral imaging sensor may comprise, for example, a hyperspectral sensor. A hyperspectral sensor may comprise a spatial scanning sensor, a spectral scanning sensor, a non-scanning sensor (snapshot), or a spatio-spectral scanning sensor.

The multispectral imaging sensor may comprise, for example, a mosaic sensor. In some cases, the mosaic sensor may comprise a plurality of cavities having different heights, which may enable the capture of different spectral wavelengths without requiring a separate optical element (e.g., one or more filters). A color camera may comprise a 2×2 array of color channels per pixel, for example, in a Bayer (RGBG) color pattern. A mosaic camera may comprise a 3×3, 4×4, 5×5, 6×6, 10×10, etc. color channels per pixel. The larger number of color channels may comprise, for example, three distinct color ranges. In some cases, the larger number of color channels may comprise a greater number of distinct color ranges.

The one or more imaging sensors may be configured for imaging in various ranges of wavelengths. For example, the imaging sensors may be configured for imaging based on light signals having a wavelength of about 400 nm to about 1 mm.

The imaging sensors disclosed herein may be configured to gather image data in three dimensions. The image sensors may be arranged to gather image data in three dimensions (e.g., two spatial dimensions and a spectral dimension) in a single exposure. This may be achieved by the image sensor having a mosaic configuration. The mosaic configuration may comprise sub-groups of light-detecting elements (i.e., sub-pixels) repeated over an array of light-detecting elements. In some cases, a filter can be provided such that a plurality of unique wavelength bands can be transmitted to the light-detecting elements in the sub-group. Each image point (made up by a single sub-group or sub-pixel) has a spectral resolution defined by the wavelength bands detected by the sub-group or sub-pixel.

In some embodiments, the system may comprise one or more imaging sensors for imaging a tissue. A tissue may comprise a portion of a surgical scene. The one or more imaging sensors may comprise at least one multispectral, hyperspectral, or mosaic imaging sensor. The multispectral sensor may be configured for (i) spectral imaging using a plurality of discrete wavelength bands and/or (ii) imaging of a plurality of spectral wavelength bands over a continuous spectral range.

The one or more imaging sensors may be configured to generate one or more images of the surgical scene based on a third set of light signals reflected, emitted, or received from the surgical scene. The third set of light signals may correspond to at least one of the first set of light signals and the second set of light signals. The one or more imaging sensors may comprise any imaging device configured to generate one or more medical images using light beams or light pulses transmitted to and reflected or emitted from a surgical scene. For example, the imaging sensors may comprise a camera, a video camera, an imaging sensor for fluorescence or autofluorescence imaging, an infrared imaging sensor, an imaging sensor for laser speckle imaging, a charge coupled device (CCD) image sensor, a complementary metal oxide semiconductor (CMOS) image sensor, a depth camera, a three-dimensional (3D) depth camera, a stereo camera, a Red Green Blue Depth (RGB-D) camera, a time-of-flight (TOF) camera, and/or an infrared camera.

### Imaging—Combined Sensor

In some cases, a single imaging sensor may be used for multiple types of imaging (e.g., any combination of fluorescence imaging, TOF depth imaging, laser speckle imaging, and/or RGB imaging). In some cases, a single imaging sensor may be used for imaging based on multiple ranges of wavelengths, each of which may be specialized for a particular type of imaging or for imaging of a particular type of biological material or physiology.

In some cases, the imaging sensors described herein may comprise an imaging sensor configured for fluorescence imaging and at least one of RGB imaging, laser speckle imaging, and TOF imaging. In some cases, the imaging sensor may be configured for fluorescence imaging and at least one of RGB imaging, perfusion imaging, and TOF imaging. In any of the embodiments described herein, the imaging sensors may be configured to see and register non-fluorescent light.

In some cases, the imaging sensors may be configured to capture fluorescence signals and laser speckle signals during alternating or different temporal slots. For example, the imaging sensor may capture fluorescence signals at a first time instance, laser speckle signals at a second time instance, fluorescence signals at a third time instance, laser speckle signals at a fourth time instance, and so on. The imaging sensor may be configured to capture a plurality of different types of optical signals at different times. The optical signals may comprise a fluorescence signal, a TOF depth signal, an RGB signal, and/or a laser speckle signal.

In other cases, the imaging sensor may be configured to simultaneously capture fluorescence signals and laser speckle signals to generate one or more medical images comprising a plurality of spatial regions. The plurality of spatial regions may correspond to different imaging modalities. For example, a first spatial region of the one or more medical images may comprise a fluorescence image based on fluorescence measurements, and a second spatial region of the one or more medical images may comprise an image based on one or more of laser speckle signals, white light or RGB signals, and TOF depth measurements.

In other cases, the imaging sensor may be configured to simultaneously capture fluorescence signals and laser speckle signals to generate one or more medical images comprising a plurality of spectral regions. The plurality of spectral regions may correspond to different imaging modalities or different pixels or different sub-pixels, as described herein. For example, a first spectral region of the one or more images may comprise a fluorescence image based on fluorescence measurements, and a second spectral region of the one or more images may comprise an image based on one or more of laser speckle signals, white light or RGB signals, and TOF depth measurements.

### Imaging—Pixels

In some embodiments, the one or more imaging sensors may comprise a plurality of pixels. In some cases, the plurality of pixels can be configured to capture different spectral wavelengths of light. The different spectral wavelengths of light may correspond to at least two of visible light, infrared light, and ultraviolet light. In some embodiments, the different spectral wavelengths of light may range from about 10 nanometers (nm) to about 1 centimeter (cm).

### Imaging—Sub-Pixels

As described elsewhere herein, in some cases, each of the one or more imaging sensors may comprise a plurality of pixels. In some cases, at least one pixel of the plurality of pixels may comprise a plurality of sub-pixels sensitive to different bands or wavelengths of light. In some cases, at least one pixel of the plurality of pixels may be configured for quantitative imaging. In some cases, a quantitative analysis of one or more features or fiducials that are detectable within the image of the tissue based on one or more light signals obtained or registered using each of the first plurality of sub-pixels and the second plurality of sub-pixels. In some cases, the quantitative imaging may be based at least in part on fluorescence imaging data (e.g., fluorescence emitted by fiducials or autofluorescing biological materials), laser speckle imaging data, RGB imaging data, and/or depth imaging data obtained using any of the imaging sensors described elsewhere herein. In some cases, the imaging sensors may comprise one or more mosaic sensors. Such mosaic sensors may be capable of multispectral imaging and/or hyperspectral imaging. The multispectral, hyperspectral, and/or mosaic imaging may enable quantitative analysis of the surgical scene being imaged or any objects or features detectable within the surgical scene.

### Imaging—Band Pass Filters

In some non-limiting embodiments, one or more band pass filters can be used to filter out one or more bands or wavelengths of light transmitted, emitted, reflected, or received from the surgical scene. In some cases, the band pass filters can be used to direct predetermined wavelengths of light or predetermined ranges of spectral wavelengths to select imaging sensors or select pixels or sub-pixels of the imaging sensors. The band pass filters can be used to enhance multispectral, hyperspectral, and/or mosaic imaging capabilities of the presently disclosed imaging systems.

### Processing—Processing Unit

In some embodiments, the system may further comprise a processing unit operatively coupled to the one or more imaging sensors. The processing unit may comprise a processor, a computing device, a logic circuit, or an FPGA.

In some cases, the processing unit may be configured to perform a quantitative analysis of one or more structures, features, and/or fiducials that are detectable within the surgical scene based on one or more light signals obtained or registered using the imaging sensors or the plurality of sub-pixels of the imaging sensors. In some cases, the one or more features or fiducials may comprise a material or substance emitting fluorescence.

In any of the embodiments described herein, the one or more light signals obtained using the plurality of sub-pixels of the imaging sensors may comprise light that is transmitted, emitted, reflected, or received from the surgical scene. In some cases, the one or more light signals obtained using the plurality of sub-pixels may comprise at least one of visible light, X-ray light, infrared light, ultraviolet light, and/or fluorescent light.

Examples of variations, embodiments, and examples of a processing unit are described further herein with respect to the section “Computer Systems.”

### Processing—Quantitative Analysis

In some embodiments, the processing unit is configured to perform a quantitative analysis of one or more features or fiducials that are detectable within the surgical scene based on one or more light signals obtained or registered using the plurality of sub-pixels of the imaging sensors described herein. The quantitative analysis may be used to numerically characterize one or more aspects or features of the surgical scene being imaged. In some cases, a quantitative analysis of one or more features or fiducials that are detectable within the image of the tissue based on one or more light signals obtained or registered using each of the first plurality of sub-pixels and the second plurality of sub-pixels

In some cases, the quantitative analysis may comprise quantifying an amount of fluorescence emitted from the surgical scene. In some cases, the quantitative analysis may comprise quantifying a concentration of a material or substance emitting the fluorescence.

In some cases, the quantitative analysis may comprise identifying or classifying one or more tissue regions in the surgical scene based on the one or more light signals. In some cases, the identification or classification may be based on one or more optical properties of the structures or features detectable within the surgical scene. The optical properties may correspond to a reflectance, an absorption, or a transmittance of light signals directed to the detectable structures or features. In some cases, the identification or classification may be based on one or more properties of the light signals received from the surgical scene and/or received by the imaging sensors disclosed herein. The one or more properties may comprise, for instance, frequency, wavelength, amplitude, phase, or phase differences/phase offsets associated with two or more light signals. The two or more light signals may comprise, for instance, a first light signal transmitted to the surgical scene and a second light signal received from the surgical scene. Alternatively, the two or more light signals may comprise, a first light signal received from the surgical scene at a first point in time and a second light signal received from the surgical scene at a second point in time.

In some cases, the quantitative analysis may comprise classification of one or more tissue regions in the surgical scene based on multispectral, hyperspectral, and/or mosaic imaging data derivable from the one or more light signals. The one or more light signals may comprise a plurality of different wavelengths. The one or more light signals may correspond to a plurality of different ranges of wavelengths. The different ranges of wavelength may correspond to a same imaging modality (e.g., visible light imaging, infrared imaging, ultraviolet imaging, X-ray imaging, fluorescence imaging, laser speckle imaging, depth imaging, etc.). In some cases, the different ranges of wavelength may correspond to different imaging modalities and/or different spectrums of light across the electromagnetic spectrum).

In some cases, the quantitative analysis may comprise determining blood oxygenation based on the one or more light signals. As used herein, blood oxygenation may generally refer to a measure of oxygen present in blood (e.g., a measure of the amount of hemoglobulin binding sites occupied by oxygen). Blood oxygenation may be expressed quantitatively as a ratio of oxygenated hemoglobin to total hemoglobin within a blood sample. The blood oxygenation may be determined in real time as the light signals are received from the surgical scene and/or processing by the processing unit.

In some cases, the quantitative analysis may comprise quantitative fluorescence analysis and/or quantitative speckle analysis based on the one or more light signals. Quantitative fluorescence analysis may comprise numerically quantifying or characterizing the surgical scene or one or more features of the surgical scene based on one or more fluorescent light signals registered using the imaging sensors described herein. Quantitative speckle analysis may comprise numerically quantifying or characterizing the surgical scene or one or more features of the surgical scene based on one or more laser speckle light signals registered using the imaging sensors described herein.

In some embodiments, the processing unit may be configured to determine an amount or a concentration of fluorescent material present in the surgical scene using spectral fitting and/or absorption spectroscopy. In some cases, spectral fitting may comprise fitting, matching, or comparing spectral data to (i) one or more baseline spectral signatures associated with the fluorescent materials when the fluorescent materials are imaged under known or predetermined imaging conditions, or (ii) one or more reference models representative of spectral data obtained under imaging conditions that are known or predetermined. In some cases, absorption spectroscopy may comprise directing a beam of electromagnetic radiation with a range of frequencies at a sample and detecting the intensity of the radiation along this range of frequencies that passes through the sample. An absorption spectrum may comprise the intensity of radiation at each of the frequencies, of the range of frequencies, that is absorbed by or passes through the sample. The amount of radiation that is absorbed by the sample at each discrete wavelength can be calculated by measuring the intensity of the radiation at discrete wavelengths that pass through the sample. The intensity (e.g., absorption values) for each discrete wavelength can be compiled into an absorption spectrum. An absorption spectrum can indicate the concentration of specific materials, structures (e.g., bonds), or elements within a sample and can be used to identify a sample or a concentration of a compound within a sample.

As described elsewhere herein, in some embodiments, the one or more light signals obtained or registered using the plurality of sub-pixels of the presently disclosed imaging sensors may comprise fluorescent light. The fluorescent light may be emitted by a fluorescent material in or near the surgical scene. Alternatively, the fluorescent light may comprise tissue autofluorescence light. Tissue autofluorescence light may comprise the natural emission of light by one or more biological structures (e.g., mitochondria and lysosomes of a cell, the extracellular matrix, nicotinamide adenine dinucleotide phosphate (NADPH) molecules, and proteins comprising an increased amount of tryptophan, tyrosine and phenylalanine). In some cases, tissue autofluorescence may interfere with detection of specific fluorescent signals because autofluorescence may cause structures other than those of interest, or the general tissue being imaged, to become visible to a fluorescence sensor. In other cases, tissue autofluorescence may aid in analysis of a tissue without artificial fluorescence markers. As an example, cellular autofluorescence may be used as an indicator of cytotoxicity without the need to add fluorescent markers. In any case, the processing unit may be configured to quantify an amount of fluorescence emitted from the surgical scene or an amount of fluorescent material present in the surgical scene. In some cases, the amount of fluorescence emitted from the surgical scene or the amount of fluorescent material present in the surgical scene may be quantified based on, for example, a radiant flux or a spectral flux of the light received from the surgical scene. As used herein, “radiant flux” may refer generally to an amount of radiant energy. The radiant energy may be emitted, reflected, and/or otherwise be directed from the surgical scene. The radiant energy may be collected, measured, and/or received by an instrument configured to measure radiant energy (e.g., a sensor operatively coupled to a processor). Radiant flux may be measured in watts (W). As used herein, “spectral flux” may refer generally to an amount of radiant energy per unit frequency or wavelength (i.e., the quantity of radiant energy distributed along a spectral range). Spectral flux may be measured in watts per hertz (W/Hz), or watts per wavelength (W/nm). In some cases, the amount of fluorescence emitted from the surgical scene or the amount of fluorescent material present in the surgical scene may be quantified based on a power or an intensity of fluorescent light emitted from the surgical scene. The amount of fluorescence emitted from the surgical scene or the amount of fluorescent material present in the surgical scene may be determined based on a quantitative analysis of one or more select wavelengths or spectral ranges of light. The amount of fluorescence emitted from the surgical scene or the amount of fluorescent material present in the surgical scene may be determined based on a quantitative analysis of the radiant flux, spectral flux, power, and/or intensity of the fluorescent light received or registered by the imaging sensors described herein. The fluorescent light received or registered by the imaging sensors may correspond to a particular set of light signals having one or more select wavelengths or a plurality of wavelengths spanning one or more spectral ranges of interest. The one or more select wavelengths and/or the one or more spectral ranges of interest may be specified by a user or an operator an imaging device comprising the imaging sensors of the present disclosure. In some cases, the one or more select wavelengths and/or the one or more spectral ranges of interest may be automatically selected by a computer based on the imaging application or the target imaging environment.

In some cases, the quantitative fluorescence imaging applications described herein may involve comparing one or more properties of the light signals transmitted to a surgical scene against one or more properties of the light signals reflected or received from the surgical scene. The one or more properties of the light signals transmitted to the surgical scene may comprise, for example, radiant flux, spectral flux, power, and/or intensity. The one or more properties of the light signals reflected or received from the surgical scene may comprise, for example, radiant flux, spectral flux, power, and/or intensity. In some cases, one or more differences in the properties of the light signals transmitted to the surgical scene and the light signals reflected or received from the surgical scene may be used to perform a quantitative analysis of the surgical scene or implement one or more of the quantitative image-based applications described herein.

### Processing—Lighting Conditions

In some cases, the processing unit may be configured to quantify an image property based on a lighting condition of the surgical scene. The lighting condition may be created or induced based on the light source used, the optical elements used to direct light from the light source to the surgical scene, the light channels used to direct the light from the light source to the surgical scene, or the method in which the light is transmitted from the light source to the surgical. In some cases, the lighting condition may be created or induced when light from the light source is transmitted from the light source to the surgical scene via a scope (e.g., an endoscope or a laparoscope). The lighting condition may comprise, for example, an illumination bias, an illumination profile, or an illumination gradient of the surgical scene. The illumination bias, illumination profile, or illumination gradient may comprise a change in lighting across a surgical scene (e.g., due to the spatial characteristics of the scene or the medium or space through which the light is transmitted before interacting with the surgical scene).

For example, the processing unit may be configured to quantify an amount of fluorescence emitted from the surgical scene or an amount of fluorescent material present in the surgical scene based on a lighting condition of the surgical scene. For example, the processing unit may be configured to quantify an amount of absorption by the surgical scene or an amount of absorbing material present in the surgical scene based on a lighting condition of the surgical scene. For example, laser speckle signal may be affected by a coherence of the laser source. The processing unit may be configured to quantify an amount of laser speckle correlation by taking into account changes in coherence based on a lighting condition of the surgical scene.

### Imaging Applications—Fluorescence Maps and Quantitative Fluorescence

In some embodiments, the processing unit may be configured to generate one or more images of the surgical scene based on image data or image signals derived from the one or more light signals. In some cases, the processing unit may be configured to generate a quantitative map of fluorescence in the surgical scene based on the image data or image signals. In one non-limiting example, the quantitative map of fluorescence may indicate an amount or concentration of fluorescence material present in one or more regions of the surgical scene. In some cases, the quantitative map of fluorescence may provide a visualization of the surgical scene with fluorescence data (e.g., fluorescent signals) overlaid on a baseline image of the surgical scene. The baseline image may comprise, for example, a visible light/RGB image of the surgical scene or a laser speckle image of the surgical scene. The quantitative map of fluorescence may provide a numerical indication of the amount or concentration of fluorescence material detected in the surgical scene. The numerical indication may be provided as part of the visualization of the surgical scene. In some cases, the numerical indication may be overlaid on a visible light/RGB image of the surgical scene or any other image overlay comprising one or more images of the surgical scene. The one or more images of the surgical scene may comprise images obtained using different types of imaging modalities. In some cases, the numerical indication may be toggled on and/or off based on user input or preference.

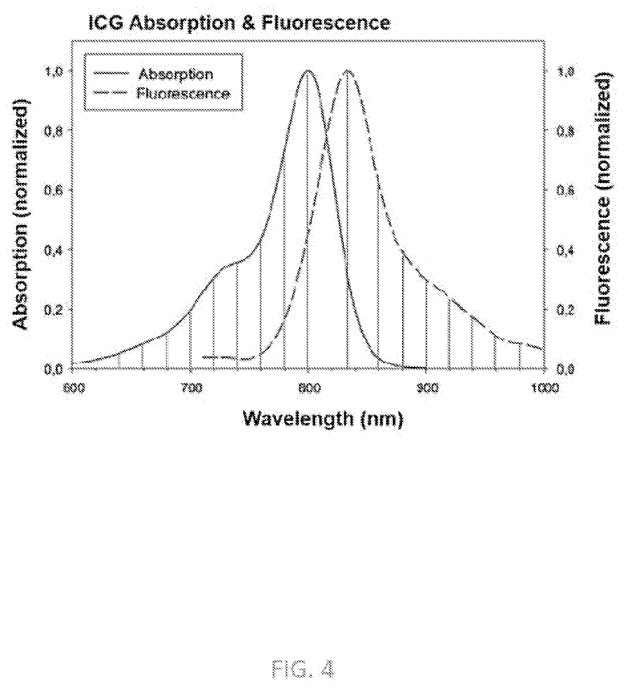

In some cases, the fluorescence maps may be generated based on quantitative fluorescence data that is derived from the light signals registered by the various sub-pixels of the imaging sensors described herein. The quantitative fluorescence data may be based on light absorption and fluorescence characteristics as a function of wavelength. An example of such light absorption and fluorescence characteristics for indocyanine green (ICG) as a function of wavelength is illustrated in FIG. 4. Another example of a fluorophore that may be used is riboflavin (B2). Another example of a fluorophore that may be used is Protoporphyrin IX (PPIX). Another example of a fluorophore is a fluorescent dye. Fluorescent compounds may include fluorescein, methylene blue, indigo carmine, patent blue, dansylamide, eosin, phloxine B, quinacrine mustard, rose Bengal, 8-anilinonaphthalene-1-sulfonic acid, Acriflavine, rhodamine dyes (e.g., rhodamine 6G), plicamycin, Oregon green, lissamine green, trypan blue, triamcinolone acetonide, bromophenol blue, brilliant blue green, infracyanine green, coumarin, cyanine, or any chemical analog or derivative thereof.

In some cases, the quantitative fluorescence data may comprise fluorescence normalization. There may be at least two general factors that affect fluorescence: distance and optical properties. Regarding distance, as the detector is further away from the source of fluorescence, the intensity of the signal may decrease. Regarding optical characteristics of the sample, different tissue types will absorb and reflect different wavelengths differently. By using an additional channel to capture the amount of excitation light that made it to each pixel and normalize the amount of fluorescence coming back to the sensor by that value, one may obtain a more reliable fluorescence reading that is less dependent on distance and optical properties. For example, the liver is quite a dark tissue, and the stomach is quite light. A stomach may appear brighter in an image regardless of the amount of dye.

In some cases, to further increase precision, optical properties may be approximated by collecting multispectral information from more channels. This may allow to approximation of the absorption and scattering coefficient for a tissue over a selected range of wavelengths. In some cases, determination of the absorption and scattering coefficient for a tissue may allow compensation for various other values extracted from the multispectral sensor (fluorescence, oxygenation, perfusion, etc.) more accurately. Based on an understanding of, for example, the absorption cross-section of a particular tissue, the value of fluorescence may be normalized across wavelength.

In some embodiments, the processing unit may be configured to perform a calibration that correlates an amount of fluorescent light detected by the one or more imaging sensors to an amount or a concentration of fluorescent material present in one or more reference regions. The amount or the concentration of fluorescent material present in the one or more reference regions may be known or estimated previously using another imaging sensor or another imaging data set derived using the same imaging sensor. Once the calibration is completed, the imaging sensors may be used to image one or more select regions of a surgical scene. The amount of fluorescent light detected by the calibrated imaging sensors may be used to determine an amount or a concentration of fluorescent material present in the one or more select regions. The one or more select regions may be different than the one or more reference regions used for calibration purposes.

In some cases, the one or more features or fiducials may comprise a fluorophore. In some cases, the fluorophore may be a porphyrin. In some cases, the porphyrin may be Protoporphyrin IX (PPIX). PPIX may comprise one or more derivatives of porphine. The one or more derivatives of porphine may comprise one or more functional groups on four pyrrole rings instead of hydrogen atoms. PPIX may comprise a porphine core. PPIX may comprise a tetrapyrrole macrocycle. PPIX may be characterized by an aromatic (e.g., mostly planar) structure. PPIX may be synthesized from acyclic precursors including a mono-pyrrole (e.g., porphobilinogen) and a tetrapyrrole (e.g., a porphyrinogen, such as uroporphyrinogen III). These acyclic precursors may be converted to protoporphyrinogen IX. Protoporphyrinogen IX may be oxidized to create PPIX. In some cases, PPIX may be synthesized from glycine and succinyl-CoA, or glutamic acid. PPIX may react with one or more metal salts to form one or more metalloprotoporphyrin IX derivatives (e.g., PPIX reacts with iron salts to form FeCl (PPIX)). PPIX may be formed by cells within a tissue. As an example, 5-aminolevulinic acid (ALA) may be exogenously administered to a tissue, where cell may metabolize ALA to form PPIX. In particular, cancer cells may metabolize a higher portion of ALA to form PPIX and create a higher accumulation of PPIX within those cancer cells.

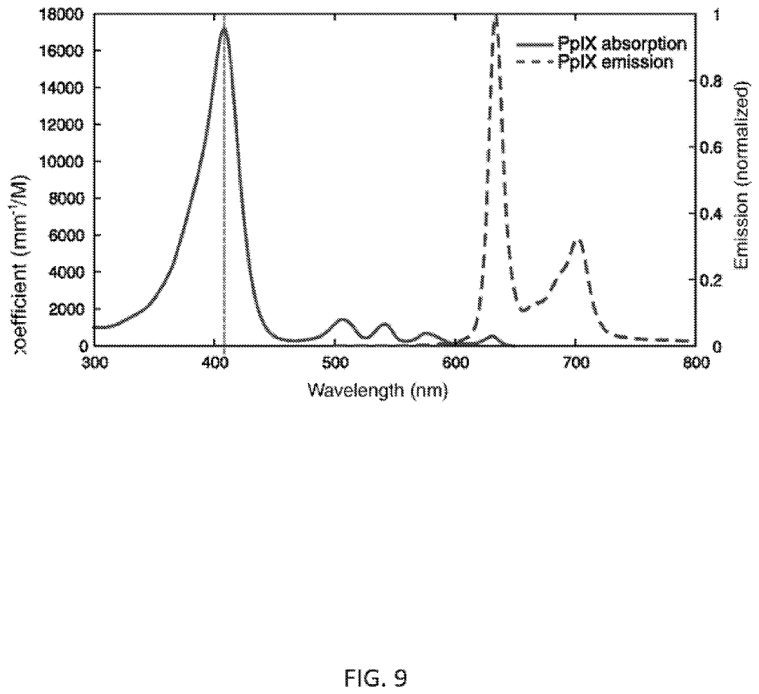

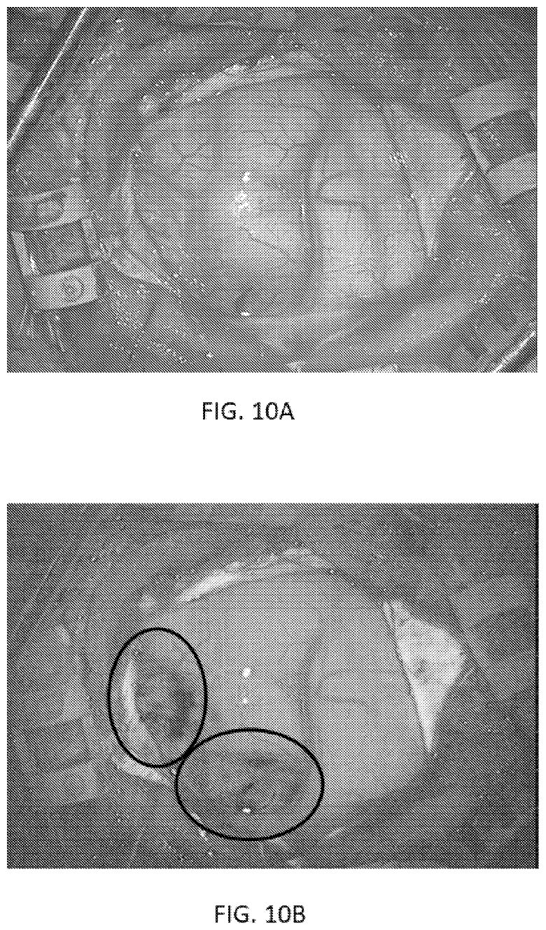

PPIX may absorb and fluoresce light. PPIX may absorb light with a wavelength between about 300 nanometers (nm) and about 650 nm. In some cases, PPIX may have a peak absorption wavelength of about 410 nm. PPIX may emit (e.g., fluoresce) light with a wavelength of about 500 nm to about 800 nm. In some cases, PPIX may have a peak emittance (e.g., fluorescence) wavelength of about 630 nm. In some cases, PPIX may have a secondary peak emittance (e.g., fluorescence) of about 700 nm. FIG. 9 illustrates absorption and emission characteristics for PPIX. FIG. 10A and FIG. 10B show an example of a surgical scene where PPIX has accumulated in one area of the tissue. FIG. 10A shows the tissue under white light. FIG. 10B shows the tissue under blue light, with cancerous regions fluorescing, circled locations in FIG. 10B. As a non-limiting example, imaging a surgical scene with blue light may cause PPIX accumulated within cancer cells of a target area to emit a wavelength that is distinguishable from the light reflected by the rest of the target area.

### Imaging Applications—Autofluorescence Applications

In some cases, the fluorophore for generating fluorescence maps and quantitative fluorescence as disclosed herein may be an injected fluorophore. In some cases, the fluorophore is a native or naturally occurring fluorophore. For example, the one or more features or fiducials may comprise a fluorophore which autofluoresces. In some cases, the fluorophore may be riboflavin (Vitamin B2). B2 may be yield natural emission in biological structures, but B2 may also be an injected fluorophore.

In some cases, the mosaic sensors described herein may be used for autofluorescence imaging based on autofluorescence signals. In some embodiments, the autofluorescence signals obtained using the systems and methods of the present disclosure may be used to visualize, detect, and/or monitor the movements of a biological material or a tissue in a target region being imaged. In some cases, the autofluorescence measurements may be used to perform temporal tracking of perfusion characteristics or other features within a surgical scene.

In some cases, the measurements and/or the light signals obtained using one or more imaging sensors of the imaging module may be used for perfusion quantification. In some cases, the measurements and/or the light signals obtained using one or more imaging sensors may be used to generate, update, and/or refine one or more perfusion maps for the surgical scene.

In some cases, the autofluorescence signals obtained using the systems and methods of the present disclosure may be used to provide a medical operator with a more accurate real-time visualization of a position or a movement of a particular point or feature within the surgical scene. In some cases, the autofluorescence signals may provide a surgeon with spatial information about the surgical scene to optimally maneuver a scope, robotic camera, robotic arm, or surgical tool relative to one or more features within the surgical scene.

In some cases, the autofluorescence signals obtained using the systems and methods of the present disclosure may be used to detect bile leaks from one or more bile ducts in the surgical scene during or after surgery. In some embodiments, the autofluorescence signals may be used to infer a hemoglobin density in tissue and to correct one or more laser speckle maps based on the inferred hemoglobin density.

In any of the embodiments described herein, the one or more images of the surgical scene may be generated based on a quantification of fluorescent light emitted from one or more biological materials in the surgical scene. In some cases, the quantification of fluorescent light can be based at least in part on (i) an amount of fluorescent light emitted from the one or more biological materials and (ii) one or more characteristics of illumination light used to capture the one or more images. The one or more characteristics of illumination light may comprise, for instance, illumination intensity, illumination gradient or bias across the surgical scene, or a distance between the surgical scene and a light source providing the illumination light.

### Imaging Applications—Blood Concentration and Oxygenation

In some embodiments, the processing unit may be configured to estimate an amount of blood in the surgical scene based on the properties or spectral characteristics of the light received or registered by the imaging sensors of the present disclosure. The amount of blood may be quantified per unit area or per unit volume. The amount of blood may estimate a volume of blood or a number of blood cells within one or more portions, sections, or areas of the surgical scene.

In some cases, the processing unit may be configured to determine an amount or a concentration of fluorophores or fluorescent material present in the surgical scene based on an estimated amount of blood in one or more portions of the surgical scene. In some cases, the processing unit may be configured to determine the amount or the concentration of fluorophores or fluorescent material present in the surgical scene based on the estimated blood concentration and one or more of the light signals received or registered by the imaging sensors of the present disclosure.

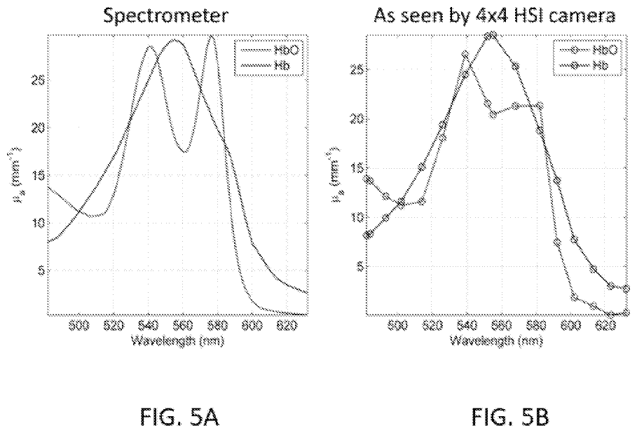

In some cases, the processing unit may be configured to determine or estimate an amount or a state of blood oxygenation based on one or more light signals received or registered by the imaging sensors of the present disclosure. In some cases, the processing unit may be configured to determine or estimate the amount or the state of blood oxygenation based on an estimated amount of blood in one or more portions of the surgical scene or an estimated amount or concentration of fluorophores or fluorescent material present in the surgical scene. In some cases, the amount or the state of blood oxygenation may be derived from information relating to blood oxyhemoglobin (HbO2) concentrations and/or blood hemoglobin (Hb) concentrations. FIG. 5A and FIG. 5B show various plots of blood oxygenation based on imaging data obtained using a spectrometer and a hyperspectral imaging camera. The imaging data may comprise information on Hb and/or HbO concentrations as a function of imaging wavelength. In any of the embodiments described herein, the blood oxygenation for a tissue in a surgical scene may be determined in real-time as the surgical scene is being imaged.

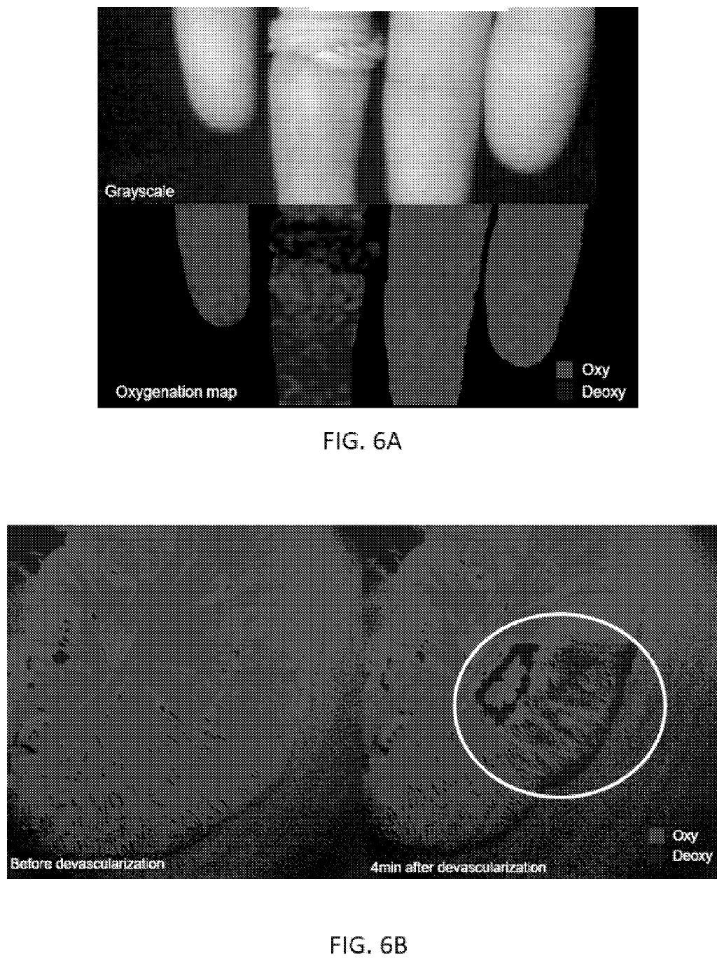

FIG. 6A and FIG. 6B show various examples of medical images obtained using the presently disclosed imaging sensors. The imaging sensors may be used to generate medical images that can be used to visual and determine blood oxygenation, and track or detect changes in blood oxygenation in real time. FIG. 6A shows a picture of four fingers of a hand with a rubber band around the ring finger. In the top panel, a normal grey scale image is shown. In the bottom images, a recolored image showing oxyhemoglobin (light grey) and deoxyhemoglobin (dark grey) is shown. As shown, the ring finger shows a significant decrease in blood oxygenation. FIG. 6B shows a picture of a pig bowel before (left) and after (right) partial devascularization. As shown in the right image, a large region of the bowel shows significant decrease in blood oxygenation.

In some cases, to further increase precision, optical properties may be approximated by collecting multispectral information from more channels. This may allow to approximation of the absorption and scattering coefficient for a tissue over a selected range of wavelengths. In some cases, determination of the absorption and scattering coefficient for a tissue may allow compensation for various other values extracted from the multispectral sensor (fluorescence, oxygenation, perfusion, etc.) more accurately. Based on an understanding of, for example, the absorption cross-section of a particular tissue, the value of absorption by oxy-and deoxy-hemoglobin may be normalized across wavelength.

In some embodiments, the processing unit may be configured to perform a calibration that correlates an amount of absorption detected by the one or more imaging sensors to an amount or a concentration of absorbing material present in one or more reference regions. The amount or the concentration of absorbing material present in the one or more reference regions may be known or estimated previously using another imaging sensor or another imaging data set derived using the same imaging sensor. Once the calibration is completed, the imaging sensors may be used to image one or more select regions of a surgical scene. The amount of absorbing light detected by the calibrated imaging sensors may be used to determine an amount or a concentration of absorbing material present in the one or more select regions. The one or more select regions may be different than the one or more reference regions used for calibration purposes.

### Imaging Applications—Quantitative Speckle

Quantitative speckle analysis may comprise numerically quantifying or characterizing the surgical scene or one or more features of the surgical scene based on one or more laser speckle light signals registered using the imaging sensors described herein. In some cases, it may be advantageous to determine an absolute velocity for blood cell movement. However, it may be difficult to calculate an absolute number because of several factors, for example, coherence of the optical source, the tissue type, etc. Regarding tissue type, the small bowel may have more red blood cells per pixel and thus a larger scattering cross section (e.g., 1.6×the large bowel). Similarly, the amount of fat in the tissue may affect the scattering cross section.

In some embodiments, the imaging sensors disclosed herein may be used to capture one or more multispectral, hyperspectral, and/or mosaic images and one or more laser speckle contrast images (LSCI). The imaging sensors may be used to capture multispectral, hyperspectral, and/or mosaic imaging data and speckle imaging data simultaneously. The speckle imaging data may comprise information on blood flow, blood oxygenation, or other characteristics of the surgical scene being imaged.

In any of the embodiments described herein, multispectral, hyperspectral, and/or mosaic information may be used to modulate one or more speckle signals obtained using one or more imaging sensors. In such cases, the multispectral, hyperspectral, and/or mosaic information can be used to update or refine LSCI images/perfusion flow maps obtained from one or more laser speckle signals. In some cases, quantitative data derived from hyperspectral or multispectral imaging data may be used to modulate or interpret one or more imaging signals associated with another type of imaging modality, such as laser speckle imaging, RGB imaging, fluorescence imaging, depth imaging, etc.).

In some cases, to further increase precision, optical properties may be approximated by collecting multispectral information from more channels. This may allow to approximation of the absorption and scattering coefficient for a tissue over a selected range of wavelengths. In some cases, determination of the absorption and scattering coefficient for a tissue may allow compensation for various other values extracted from the multispectral sensor (fluorescence, oxygenation, perfusion, etc.) more accurately. Based on an understanding of, for example, the scattering cross-section of a particular tissue, the value of laser speckle may be normalized across wavelength. In another example, by using optical property correction to estimate the amount of blood present at every pixel, it may be possible to determine the amount of speckle signal that results from blood cell movement. This may allow quantification of the perfusion signal using absolute velocity units.

In some embodiments, the processing unit may be configured to perform a calibration that correlates an amount of speckle detected by the one or more imaging sensors to an amount or a concentration of scattering material present in one or more reference regions. The amount or the concentration of scattering material present in the one or more reference regions may be known or estimated previously using another imaging sensor or another imaging data set derived using the same imaging sensor. Once the calibration is completed, the imaging sensors may be used to image one or more select regions of a surgical scene. The amount of speckle light detected by the calibrated imaging sensors may be used to determine an amount or a concentration of scattering material present in the one or more select regions. The one or more select regions may be different than the one or more reference regions used for calibration purposes.

### Imaging Applications—Tissue Classification

In some cases, the processing unit may be configured to perform object or feature classification for one or more images that are derived using light signals registered or received by the one or more imaging sensors described herein. Such classification can be performed based on a multispectral, hyperspectral, and/or mosaic imaging data set that is obtained using the one or more imaging sensors. In some cases, the one or more images containing the objects or features to be classified may comprise RGB or visible light images. The objects or features in the RGB or visible light images may be classified using additional imaging data (e.g., speckle imaging data, depth imaging data, fluorescence imaging data, etc.). The additional imaging data may comprise raw, unprocessed light signals obtained using the imaging sensors and/or any quantitative or qualitative data derived from processing or analyzing the raw light signals. The additional imaging data may be obtained using different imaging modalities or variations combinations of imaging modalities. The additional imaging data may be obtained using multiple imaging wavelengths and/or multiple spectral ranges for imaging.

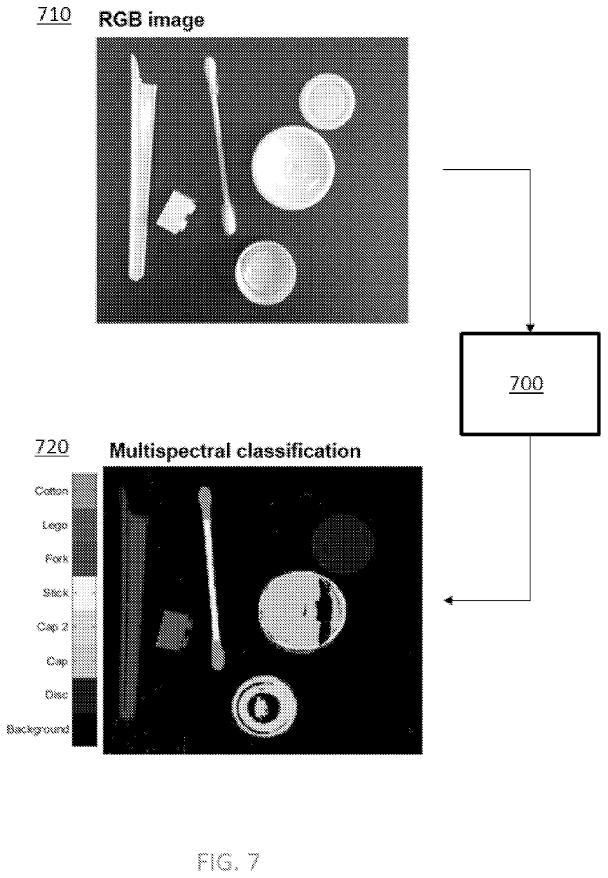

FIG. 7 illustrates an example of a processing unit 700 configured to perform multispectral classification 720 based on a baseline RGB image 710 and additional imaging data derived from the light signals registered using a multispectral, hyperspectral, or imaging sensor. The baseline RGB image may be generated from imaging data derived from light signals registered using the same multispectral, hyperspectral, or mosaic imaging sensor. Alternatively, baseline RGB image may be generated from imaging data derived from light signals registered using a separate imaging sensor. The separate imaging sensor may comprise, for example, an RGB imaging sensor, a camera, or another multispectral, hyperspectral, or mosaic imaging sensor.

In some cases, the method further comprises a classifier model used to detect critical structures in an image. The critical structures may comprise the detection of anomalous tissue, a map of optical properties, a tissue classification, etc. In some cases, multispectral, hyperspectral, and/or mosaic images may be inputs to a classifier model. For example, multispectral, hyperspectral, and/or mosaic images may comprise depth maps, quantitative fluorophore concentration maps, and/or quantitative speckle maps which may be the product of multispectral, hyperspectral, and/or mosaic images as described herein.

In some cases, the classifier comprise a computer implemented model. In some cases, the classifier is a trained classifier. In some cases, the classifier is a machine learning algorithm. For example, the machine learning algorithm may use multispectral, hyperspectral, and/or mosaic images as inputs. The machine algorithm may be used to detect critical structures in an image. The critical structures may comprise the detection of anomalous tissue, a map of optical properties, a tissue classification, etc. In some cases, multispectral, hyperspectral, and/or mosaic images may comprise depth maps, quantitative fluorophore concentration maps, and/or quantitative speckle maps which may be the product of multispectral, hyperspectral, and/or mosaic images may be inputs the classifier. In some cases, inputs may comprise one or more extracted image features such as regions of interest within an image.

In some cases, the machine learning algorithm comprises one or more of linear regressions, logistic regressions, classification and regression tree algorithms, support vector machines (SVMs), naive Bayes, K-nearest neighbors, random forest algorithms, boosted algorithms such as XGBoost and LightGBM, neural networks, convolutional neural networks, and recurrent neural networks. In some embodiments, the machine learning algorithm is a supervised learning algorithm, an unsupervised learning algorithm, or a semi-supervised learning algorithm.

Machine learning algorithms may be used in order to make predictions using a set of parameters. One class of machine learning algorithms, artificial neural networks (ANNs), may comprise a portion of the classifier model. For example, feedforward neural networks (such as convolutional neural networks or CNNs) and recurrent neural networks (RNNs) may be used. A neural network binary classifier may be trained by comparing predictions made by its underlying machine learning model to a ground truth. An error function calculates a discrepancy between the predicted value and the ground truth, and this error is iteratively backpropagated through the neural network over multiple cycles, or epochs, in order to change a set of weights that influence the value of the predicted output. Training ceases when the predicted value meets a convergence condition, such as obtaining a small magnitude of calculated error. Multiple layers of neural networks may be employed, creating a deep neural network. Using a deep neural network may increase the predictive power of a neural network algorithm. In some cases, a machine learning algorithm using a neural network may further include Adam optimization (e.g., adaptive learning rate), regularization, etc. The number of layers, the number of nodes within the layer, a stride length in a convolutional neural network, a padding, a filter, etc. may be adjustable parameters in a neural network.

Additional machine learning algorithms and statistical models may be used in order to obtain insights from the parameters disclosed herein. Additional machine learning methods that may be used are logistic regressions, classification and regression tree algorithms, support vector machines (SVMs), naive Bayes, K-nearest neighbors, and random forest algorithms. These algorithms may be used for many different tasks, including data classification, clustering, density estimation, or dimensionality reduction. Machine learning algorithms may be used for active learning, supervised learning, unsupervised learning, or semi-supervised learning tasks. In this disclosure, various statistical, machine learning, or deep learning algorithms may be used to generate an output based on the set of parameters.

A machine learning algorithm may use a supervised learning approach. In supervised learning, the algorithm can generate a function or model from training data. The training data can be labeled. The training data may include metadata associated therewith. Each training example of the training data may be a pair consisting of at least an input object and a desired output value. A supervised learning algorithm may require the user to determine one or more control parameters. These parameters can be adjusted by optimizing performance on a subset, for example, a validation set, of the training data. After parameter adjustment and learning, the performance of the resulting function/model can be measured on a test set that may be separate from the training set. Regression methods can be used in supervised learning approaches.

In some embodiments, the supervised machine learning algorithms can include but not being limited to neural networks, support vector machines, nearest neighbor interpolators, decision trees, boosted decision stump, boosted version of such algorithms, derivatives versions of such algorithms, or their combinations. In some embodiments, the machine learning algorithms can include one or more of: a Bayesian model, decision graphs, inductive logic programming, Gaussian process regression, genetic programming, kernel estimators, minimum message length, multilinear subspace learning, naive Bayes classifier, maximum entropy classifier, conditional random field, minimum complexity machines, random forests, ensembles of classifiers, and a multicriteria classification algorithm.

A machine learning algorithm may use a semi-supervised learning approach. Semi-supervised learning can combine both labeled and unlabeled data to generate an appropriate function or classifier.

In some embodiments, a machine learning algorithm may use an unsupervised learning approach. In unsupervised learning, the algorithm may generate a function/model to describe hidden structures from unlabeled data (i.e., a classification or categorization that cannot be directed observed or computed). Since the examples given to the learner are unlabeled, there is no evaluation of the accuracy of the structure that is output by the relevant algorithm.

Approaches to unsupervised learning include clustering, anomaly detection, and neural networks.

A machine learning algorithm may use a reinforcement learning approach. In reinforcement learning, the algorithm can learn a policy of how to act given an observation of the world. Every action may have some impact in the environment, and the environment can provide feedback that guides the learning algorithm.

For example, a machine learning algorithm may be used to identify patterns of similar structure between images from different imaging modalities. Areas of high blood flow may appear as distinct from the surrounding tissue in laser speckle date, blood oxygenation images, and quantitative fluorescence images. By identifying analogously behaving regions in various imaging modalities, a machine learning algorithm may be useful in determining areas of clinical relevance.

### Surgical Procedures

The systems and methods of the present disclosure may be implemented to perform multispectral imaging for various types of surgical procedures. The surgical procedure may comprise one or more general surgical procedures, neurosurgical procedures, orthopedic procedures, and/or spinal procedures. In some cases, the one or more surgical procedures may comprise colectomy, cholecystectomy, appendectomy, hysterectomy, thyroidectomy, and/or gastrectomy. In some cases, the one or more surgical procedures may comprise hernia repair, and/or one or more suturing operations. In some cases, the one or more surgical procedures may comprise bariatric surgery, large or small intestine surgery, colon surgery, hemorrhoid surgery, and/or biopsy (e.g., liver biopsy, breast biopsy, tumor, or cancer biopsy, etc.).

In some embodiments, the one or more images of the surgical scene may be usable to detect bile leaks from one or more bile ducts in the surgical scene during or after surgery. In some embodiments, the one or more images of the surgical scene may be usable to infer a hemoglobin density in tissue and to correct one or more laser speckle maps based on the inferred hemoglobin density.

In some cases, quantitative measurements based on multispectral images may allow for statistical estimates of patient outcomes based on the data. For example, quantitative measurements of tissue oxygenation may allow for a calculation of a percent likelihood that a tissue becomes necrotic after surgery. For example, quantitative measurements of blood velocity from laser speckle data may allow for a calculation of a percent likelihood that a tissue becomes necrotic after surgery.

In some cases, quantitative measurements may allow for more accurate indications to a surgeon of the efficacy of mid-surgical operations. For example, an absolute measurement of tissue oxygenation or blood flow may provide an indication of the success of blood vessel clamping during a surgical operation. A visual indication of the degree of success in tissue clamping may reduce the likelihood of a bleed during an operation and may generally improve patient safety and outcomes.

### System

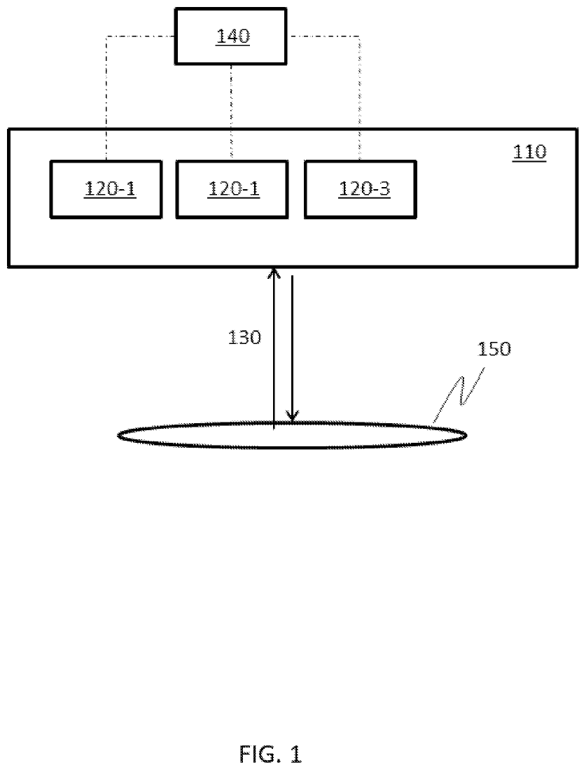

FIG. 1 illustrates an example system for multispectral, hyperspectral, and/or mosaic imaging. The system may comprise an imaging module 110 for medical imaging of a surgical scene. The medical imaging may be performed using one or more light signals generated using one or more light sources. The one or more light signals may be used to perform multispectral, hyperspectral, and/or mosaic imaging.

In some embodiments, the system may comprise one or more imaging sensors as described elsewhere herein. In some cases, the one or more imaging sensors may comprise a single imaging sensor or a plurality of imaging sensors (e.g., two or more imaging sensors). As shown in FIG. 1, in some cases the system may comprise imaging sensors 120-1, 120-2, 120-3. The one or more imaging sensors 120-1, 120-2, 120-3 may comprise, for example, a mosaic sensor and/or any other imaging sensor configured for multispectral, hyperspectral, and/or mosaic imaging. In some cases, the one or more imaging sensors may comprise two or more imaging sensors configured for different types of imaging (e.g., fluorescence imaging, autofluorescence imaging, time of flight imaging, RGB or visible light imaging, and/or laser speckle imaging).

The imaging module 110 may be configured to receive one or more signals from a surgical scene 150. The one or more signals from the surgical scene 150 may comprise one or more optical signals 130. The one or more optical signals 130 may correspond to one or more light waves, light pulses, or light beams generated using the one or more light sources. The one or more optical signals 130 may be generated when the one or more light waves, light pulses, or light beams generated using the one or more light sources are transmitted to the surgical scene 150. The optical signals 130 may be generated based on an interaction between (i) a feature, a marker, or a biological material or tissue in the surgical scene and (ii) the light waves, light pulses, or light beams generated using the one or more light sources. Such interaction may comprise, for example, transmission of light, reflection of light, or absorption of light by the feature, the marker, or the biological material or tissue. In some cases, the light waves, light pulses, or light beams generated using the one or more light sources may cause the feature, the marker, or the biological material to fluoresce. In some cases, the one or more light waves, light pulses, or light beams generated using the one or more light sources may be transmitted to the surgical scene 150 via a scope (e.g., a laparoscope). In some cases, the optical signals 130 from the surgical scene 150 may be transmitted back to the imaging module 110 via the scope. The optical signals (or a subset thereof) may be directed to the appropriate imaging sensor (e.g., using an optical element such as a beam splitter or a dichroic mirror).

In some embodiments, the system may comprise a processing unit 140 as described elsewhere herein. The imaging sensors 120-1, 120-2, 120-3 may be operatively coupled to the processing unit 140. The processing unit 140 may be configured to generate one or more images of the surgical scene 150 based on the optical signals 130 received at the one or more imaging sensors 120-1, 120-2, 120-3. In some cases, the processing unit 140 may be provided separately from the imaging module 110. In other cases, the processing unit 140 may be integrated with or provided as a component of the imaging module 110.

### Light Sources

As described above, one or more light sources may be used to generate light signals for multispectral, hyperspectral, and/or mosaic imaging. The system may comprise a plurality of light sources for illuminating a surgical scene. In some cases, the plurality of light sources may comprise (i) a first light source configured to generate a first set of light signals for fluorescence imaging and (ii) a second light source configured to generate a second set of light signals for at least one of RGB imaging, laser speckle imaging, and depth imaging. In some cases, the plurality of light sources may comprise at least one of a white light source, a laser speckle light source, and a fluorescence excitation light source. In other cases, the plurality of light sources may not or need not comprise a white light source, a laser light source, or a fluorescence excitation light source.

### Beams/Pulses

In any of the embodiments described herein, the plurality of light sources may be configured to generate one or more light beams. In such cases, the plurality of light sources may be configured to operate as a continuous wave light source. A continuous wave light source may be a light source that is configured to produce a continuous, uninterrupted beam of light with a stable output power.

In some cases, the plurality of light sources may be configured to continuously emit pulses of light and/or energy at predetermined intervals. In such cases, the light sources may only be switched on for limited time intervals, and may alternate between a first power state and a second power state. The first power state may be a low power state or an OFF state. The second power state may be a high power state or an ON state.

Alternatively, the plurality of light sources may be operated in a continuous wave mode, and the one or more light beams generated by the plurality of light sources may be chopped (i.e., separated or discretized) into a plurality of light pulses using a mechanical component (e.g., a physical object or shuttering mechanism) that blocks the transmission of light at predetermined intervals. The mechanical component may comprise a movable plate that is configured to obstruct an optical path of one or more light beams generated by the plurality of light sources, at one or more predetermined time periods.

### Fluorescence Excitation Light Source

In some cases, the light sources may comprise a fluorescence excitation light source. The fluorescence excitation light source may be used for fluorescence imaging. As used herein, fluorescence imaging may refer to the imaging of any fluorescent materials (e.g., auto fluorescing biological materials such as tissues or organs) or fluorescing materials (e.g., dyes comprising a fluorescent substance like fluorescein, coumarin, cyanine, rhodamine, or any chemical analog or derivative thereof). The fluorescence excitation light source may be configured to generate a fluorescence excitation light beam. The fluorescence excitation light beam may cause a tissue or a fluorescent dye (e.g., indocyanine green) to fluoresce (i.e., emit light). The fluorescence excitation light beam may have a wavelength that ranges from about 450 nanometers (nm) to about 500 nanometers (nm). The fluorescence excitation light beam may be emitted onto a target tissue or biological material with native autofluorescence properties. In some cases, the target region may emit fluorescent light signals with a wavelength that is greater than about 500 nanometers (nm).

In some cases, the fluorescence excitation light source may be configured to generate blue light having a wavelength of about 470 nm to excite autofluorescing tissue, which can then emit light having wavelengths of at least about 500 nm. In some cases, a filter may be used to block the 470 nm light from the camera so that the autofluorescence imaging sensor only sees the 500+ nm light emitted by the autofluorescing tissue.

In some cases, the fluorescence excitation wavelength may be configured to generate light having a wavelength ranging from about 300 nm to about 500 nm. In some cases, the excitation wavelength may be directed to autofluorescing tissue. Light emitted from autofluorescing tissue may have a wavelength that is greater than the excitation wavelength used to excite the tissue. In some cases, the emitted light may have a wavelength that is greater than 300 nm. In some cases, the wavelength of light emitted by the autofluorescing tissue may be greater than 350 nm.

In some cases, the fluorescence excitation wavelength may be used for UV imaging. As an example, the excitation wavelength may be about 350 nm and a collected light wavelength may have a wavelength of about 400 nm.

In some cases, the fluorescence excitation light source may be configured to generate one or more light pulses, light beams, or light waves. In some cases, the one or more light pulses, light beams, or light waves may be used for tissue autofluorescence imaging and/or other types of imaging (e.g., RGB imaging, laser speckle imaging, or time of flight (TOF) imaging).

In some embodiments, the fluorescence excitation light source may be used to generate a plurality of fluorescence excitation light pulses. In such cases, the fluorescence excitation light source may be pulsed (i.e., switched ON and OFF at one or more predetermined intervals). In some cases, such pulsing may be synced to an opening and/or a closing of one or more camera shutters for synchronized autofluorescence imaging.

In some embodiments, the fluorescence excitation light source may be used to generate a continuous light beam. In some cases, the fluorescence excitation light source may be continuously ON, and a property of the fluorescence excitation light may be modulated. For example, the continuous light beam may undergo an amplitude modulation. The amplitude modulated light beam may be used to obtain one or more measurements based on a phase difference between the fluorescence excitation light and the autofluorescence light emitted and received from the tissue. The fluorescence measurements may be computed based at least in part on a phase shift observed between the fluorescence excitation light directed to the target region and the autofluorescence light emitted and received from the target region. In other cases, when the fluorescence excitation light source is used to generate a continuous light beam, one or more movable mechanisms (e.g., an optical chopper or a physical shuttering mechanism such as an electromechanical shutter or gate) may be used to generate a series of fluorescence excitation pulses from the continuous light beam. The plurality of fluorescence excitation light pulses may be generated by using a movement of the electromechanical shutter or gate to chop, split, or discretize the continuous light beam into the plurality of fluorescence excitation light pulses. One advantage of having the fluorescence excitation light beam continuously on is that there are no delays in ramp-up and/or ramp-down (i.e., no delays associated with powering the beam on and off).

In some embodiments, the fluorescence excitation light source may be located remote from a scope and operatively coupled to the scope via a light guide. For example, the fluorescence excitation light source may be located on or attached to a surgical tower. In other embodiments, the fluorescence excitation light source may be located on the scope and configured to provide the fluorescence excitation light to the scope via a scope-integrated light guide. The scope-integrated light guide may comprise a light guide that is attached to or integrated with a structural component of the scope. The light guide may comprise a thin filament of a transparent material, such as glass or plastic, which is capable of transmitting light signals through successive internal reflections. Alternatively, the fluorescence excitation light source may be configured to provide the fluorescence excitation light to the target region via one or more secondary illuminating scopes. In such cases, the system may comprise a primary scope that is configured receive and direct light generated by other light sources (e.g., a white light source, a laser speckle light source, and/or a TOF light source). The one or more secondary illuminating scopes may be different than the primary scope. The one or more secondary illuminating scopes may comprise a scope that is separately controllable or movable by a medical operator or a robotic surgical system. The one or more secondary illuminating scopes may be provided in a first set of positions or orientations that is different than a second set of positions or orientations in which the primary scope is provided. In some cases, the fluorescence excitation light source may be located at a tip of the scope. In other cases, the fluorescence excitation light source may be attached to a portion of the surgical subject's body. The portion of the surgical subject's body may be proximal to the target region being imaged using the medical imaging systems of the present disclosure. In any of the embodiments described herein, the fluorescence excitation light source may be configured to illuminate the target region through a rod lens. The rod lens may comprise a cylindrical lens configured to enable beam collimation, focusing, and/or imaging. In some cases, the fluorescence excitation light source may be configured to illuminate the target region through a series or a combination of lenses (e.g., a series of relay lenses).

In some embodiments, the system may comprise a fluorescence excitation light source configured to transmit the first set of light signals to the surgical scene. The fluorescence excitation light source may be configured to generate and transmit one or more fluorescence excitation light pulses to the surgical scene. In some cases, the fluorescence excitation light source may be configured to provide a spatially varying illumination to the surgical scene. In some cases, the fluorescence excitation light source may be configured to provide a temporally varying illumination to the surgical scene. In some cases, the timing of the opening and/or closing of one or more shutters associated with one or more imaging units may be adjusted based on the spatial and/or temporal variation of the illumination. In some cases, the image acquisition parameters for the one or more imaging units may be tuned based on the surgical application (e.g., type of surgical procedure), a scope type, or a cable length. In some cases, the fluorescence measurement and acquisition scheme may be tuned based on a distance between the surgical scene and one or more components of the fluorescence imaging systems disclosed herein.

In some cases, the fluorescence excitation light source may be configured to adjust an intensity of the first set of light signals. In some cases, the fluorescence excitation light source may be configured to adjust a timing at which the first set of light signals is transmitted. In some cases, the fluorescence excitation light source may be configured to adjust an amount of light directed to one or more regions in the surgical scene. In some cases, the fluorescence excitation light source may be configured to adjust one or more properties of the first set of light signals based on a type of surgical procedure, a type of tissue in the surgical scene, a type of scope through which the light signals are transmitted, or a length of a cable used to transmit the light signals from the fluorescence excitation light source to a scope. The one or more properties may comprise, for example, a pulse width, a pulse repetition frequency, or an intensity.

In some cases, the fluorescence excitation light source may be configured to generate a plurality of light pulses, light beams, or light waves for fluorescence imaging. In some cases, the fluorescence excitation light source may be configured to generate light pulses, light beams, or light waves having multiple different wavelengths or ranges of wavelengths.

### Light Modulator

In some embodiments, the system may further comprise a light modulator. The light modulator may be configured to adjust one or more properties (e.g., illumination intensity, direction of propagation, travel path, etc.) of the fluorescence excitation light generated using the fluorescence excitation light source. In some cases, the light modulator may comprise a diverging lens that is positioned along a light path of the fluorescence excitation light. The diverging lens may be configured to modulate an illumination intensity of the fluorescence excitation light across the target region. In other cases, the light modulator may comprise a light diffusing element that is positioned along a light path of the fluorescence excitation light. The light diffusing element may likewise be configured to modulate an illumination intensity of the fluorescence excitation light across the target region. Alternatively, the light modulator may comprise a beam steering element configured to illuminate the target region and one or regions proximal to the target region. The beam steering element may be used to illuminate a greater proportion of a scene comprising the target region with the autofluorescence excitation light. In some cases, the beam steering element may comprise a lens or a mirror (e.g., a fast steering mirror).

### Fluorescence Excitation Parameter Optimizer

In some embodiments, the system may further comprise a parameter optimizer configured to adjust one or more pulse parameters and one or more camera/imaging parameters, based at least in part on a desired application, tissue type, scope type, or procedure type. The one or more fluorescence measurements obtained using the fluorescence imaging sensor may be based at least in part on the one or more pulse parameters and the one or more camera parameters. For example, the parameter optimizer may be used to implement a first set of pulse parameters and camera parameters for a first procedure, and to implement a second set of pulse parameters and camera parameters for a second procedure. The parameter optimizer may be configured to adjust the one or more pulse parameters and/or the one or more camera parameters to improve a resolution, accuracy, or tolerance of fluorescence sensing, and to increase the signal to noise ratio for fluorescence applications. In some cases, the parameter optimizer may be configured to determine the actual or expected performance characteristics of the fluorescence sensing system based on a desired selection or adjustment of one or more pulse parameters or camera parameters. Alternatively, the parameter optimizer may be configured to determine a set of pulse parameters and camera parameters required to achieve a desired resolution, accuracy, or tolerance for a desired biological material or tissue type or surgical operation.

In some cases, the parameter optimizer may be configured to adjust the one or more pulse parameters and the one or more camera parameters in real time. In other cases, the parameter optimizer may be configured to adjust the one or more pulse parameters and the one or more camera parameters offline. In some cases, the parameter optimizer may be configured to adjust the one or more pulse parameters and/or camera parameters based on a feedback loop. The feedback loop may be implemented using a controller (e.g., a programmable logic controller, a proportional controller, a proportional integral controller, a proportional derivative controller, a proportional integral derivative controller, or a fuzzy logic controller). In some cases, the feedback loop may comprise a real-time control loop that is configured to adjust the one or more pulse parameters and/or the one or more camera parameters based on a temperature of the autofluorescence excitation light source or the fluorescence imaging sensor. In some embodiments, the system may comprise an image post processing unit configured to update the fluorescence measurements based on an updated set of fluorescence measurements obtained using the one or more adjusted pulse parameters or camera parameters.

The parameter optimizer may be configured to adjust one or more pulse parameters. The one or more pulse parameters may comprise, for example, an illumination intensity, a pulse width, a pulse shape, a pulse count, a pulse on/off level, a pulse duty cycle, a fluorescence excitation light pulse wavelength, a light pulse rise time, and a light pulse fall time. The illumination intensity may correspond to an amount of power needed to provide a detectable light signal during a procedure. The pulse width may correspond to a duration of the pulses. The system may require a fluorescence excitation pulse of some minimal or maximal duration to guarantee a certain acceptable resolution. The pulse shape may correspond to a phase, an amplitude, or a period of the pulses. The pulse count may correspond to a number of pulses provided within a predetermined time period. Each of the pulses may have at least a predetermined amount of power (in Watts) in order to enable fluorescence measurements with reduced noise. The pulse on/off level may correspond to a pulse duty cycle. The pulse duty cycle may be a function of the ratio of pulse duration or pulse width (PW) to the total period (T) of the pulse waveform. The fluorescence excitation pulse wavelength may correspond to a wavelength of the fluorescence excitation light from which the fluorescence excitation light pulse is derived. The fluorescence excitation pulse wavelength may be predetermined, or adjusted accordingly for each desired fluorescence imaging application. The pulse rise time may correspond to an amount of time for the amplitude of a pulse to rise to a desired or predetermined peak pulse amplitude. The pulse fall time may correspond to an amount of time for the peak pulse amplitude to fall to a desired or predetermined threshold value. The pulse rise time and/or the pulse fall time may be modulated to meet a certain threshold value. In some cases, the fluorescence excitation light source may be pulsed from a lower power mode (e.g., 50%) to higher power mode (e.g., 90%) to minimize rise time. In some cases, a movable plate or other mechanical object (e.g., a shutter) may be used to chop a continuous fluorescence excitation light beam into a plurality of fluorescence excitation light pulses, which can also minimize or reduce pulse rise time.

The parameter optimizer may be configured to adjust one or more camera/imaging parameters. The camera parameters may include, for example, a number of shutters, shutter timing, shutter overlap, shutter spacing, and shutter duration. As used herein, a shutter may refer to a physical shutter and/or an electronic shutter. A physical shutter may comprise a movement of a shuttering mechanism (e.g., a leaf shutter or a focal-plane shutter of an imaging device or imaging sensor) in order to control exposure of light to the imaging device or imaging sensor. An electronic shutter may comprise turning one or more pixels of an imaging device or imaging sensor ON and/or OFF to control exposure. The number of shutters may correspond to a number of times in a predetermined time period during which the autofluorescence imaging sensor or camera is shuttered open to receive fluorescence light pulses emitted from the target region. In some cases, two or more shutters may be used for a fluorescence excitation light pulse. Temporally spaced shutters can be used to determine or detect one or more features in the target region. In some cases, a first shutter may be used for a first pulse (e.g., an outgoing pulse), and a second shutter may be used for a second pulse (e.g., an incoming pulse). Shutter timing may correspond to a timing of shutter opening and/or shutter closing based on a timing of when a pulse is transmitted and/or received. The opening and/or closing of the shutters may be adjusted to capture one or more fluorescence pulses. In some cases, the shutter timing may be adjusted based on a path length of the fluorescence pulses or a target region of interest. Shutter timing modulation may be implemented to minimize the duty cycle of fluorescence excitation light source pulsing and/or camera shutter opening and closing, which can enhance the operating conditions of the fluorescence excitation light source and improve hardware longevity (e.g., by limiting or controlling the operating temperature). Shutter overlap may correspond to a temporal overlap of two or more shutters. Shutter overlap may increase peak Rx power at short pulse widths where peak power is not immediately attained. Shutter spacing may correspond to the temporal spacing or time gaps between two or more shutters. Shutter spacing may be adjusted to time the camera shutters for fluorescence imaging to receive the beginning and/or the end of the pulse. Shutter spacing may be optimized to increase the accuracy of fluorescence measurements at decreased Rx power. Shutter duration may correspond to a length of time during which the fluorescence imaging sensor or camera is shuttered open to receive fluorescence light signals emitted from the target region. Shutter duration may be modulated to minimize noise associated with a received fluorescence light signal, and to ensure that the imaging sensor or camera receives a minimum amount of light needed for fluorescence imaging applications.

In some cases, hardware may be interchanged or adjusted in addition to or in lieu of software-based changes to pulse parameters and camera parameters, in order to achieve the desired fluorescence imaging capabilities for a particular application or type of tissue or biological material.

### White Light Source

In some cases, the second light source may comprise a white light source. The white light source may be configured to generate one or more light beams or light pulses having one or more wavelengths that lie within the visible spectrum. The white light source may comprise a lamp (e.g., an incandescent lamp, a fluorescent lamp, a compact fluorescent lamp, a halogen lamp, a metal halide lamp, a fluorescent tube, a neon lamp, a high intensity discharge lamp, or a low pressure sodium lamp), a light bulb (e.g., an incandescent light bulb, a fluorescent light bulb, a compact fluorescent light bulb, or a halogen light bulb), and/or a light emitting diode (LED). The white light source may be configured to generate a white light beam. The white light beam may be a polychromatic emission of light comprising one or more wavelengths of visible light. The one or more wavelengths of light may correspond to a visible spectrum of light. The one or more wavelengths of light may have a wavelength between about 400 nanometers (nm) and about 700 nanometers (nm). In some cases, the white light beam may be used to generate an RGB image of a target region. In some cases, the one or more wavelengths of light may have a wavelength between about 400 nm and about 1000 nm (e.g., near infra-red range). In some cases, the one or more wavelengths may have a wavelength between about 300 nm and about 1000 nm. In some cases, the one or more wavelengths may have a wavelength less than 300 nm. In some cases the one or more wavelengths may have a wavelength greater than 1000 nm.

### Laser Speckle Light Source

In some cases, the second light source may comprise a laser speckle light source. The laser speckle light source may comprise one or more laser light sources. The laser speckle light source may comprise one or more light emitting diodes (LEDs) or laser light sources configured to generate one or more laser light beams with a wavelength between about 700 nanometers (nm) and about 1 millimeter (mm). In some cases, the one or more laser light sources may comprise two or more laser light sources that are configured to generate two or more laser light beams having different wavelengths. The two or more laser light beams may have a wavelength between about 700 nanometers (nm) and about 1 millimeter (mm). The laser speckle light source may comprise an infrared (IR) laser, a near-infrared laser, a short-wavelength infrared laser, a mid-wavelength infrared laser, a long-wavelength infrared laser, and/or a far-infrared laser. The laser speckle light source may be configured to generate one or more light beams or light pulses having one or more wavelengths that lie within the invisible spectrum. The laser speckle light source may be used for laser speckle imaging of a target region.

### Autofluorescence Light Signals

In some embodiments, the first set of light signals for fluorescence imaging may be configured to excite one or more biological materials in the surgical scene, thereby causing the one or more biological materials to emit one or more fluorescence signals that are detectable by the one or more imaging devices. The one or more fluorescence signals emitted by the one or more biological materials may have a different wavelength than the first set of light signals used to excite the one or more biological materials. In some cases, the first set of light signals for fluorescence imaging may have a wavelength ranging from about 450 nanometers (nm) to about 500 nanometers (nm). In some cases, the first set of light signals for fluorescence imaging may have a wavelength of about 470 nanometers (nm). In some cases, the one or more fluorescence signals emitted by the one or more biological materials may have a wavelength of at least about 500 nanometers (nm). In some embodiments, the first set of light signals may not cause blood in the surgical scene to fluoresce. In some embodiments, the first set of light signals may cause one or more tissue regions in the surgical scene to fluoresce.

In some cases, the first set of light signals for fluorescence imaging may have a wavelength ranging from about 300 nanometers (nm) to about 500 nanometers (nm). In some cases, the first set of light signals for fluorescence imaging may have a wavelength of about 350 nanometers (nm). In some cases, the one or more fluorescence signals emitted by the one or more biological materials may have a wavelength of at least about 350 nanometers (nm). In some embodiments, the first set of light signals may not cause blood in the surgical scene to fluoresce. In some embodiments, the first set of light signals may cause one or more tissue regions in the surgical scene to fluoresce.

As described above, a plurality of light sources may be used to illuminate a surgical scene. In some cases, the plurality of light sources may comprise (i) a first light source configured to generate a first set of light signals for fluorescence imaging and (ii) a second light source configured to generate a second set of light signals for at least one of RGB imaging, laser speckle imaging, and depth imaging.

In some cases, the one or more images generated using the one or more imaging devices may comprise one or more fluorescence images of the one or more biological materials. The one or more biological materials may comprise, for example, a tissue. In some cases, the one or more biological materials may comprise bile, urine, fat, connective tissue, or cauterized tissue. In any of the embodiments described herein, the one or more fluorescence images may be generated without the use of any dyes or other fluorescent markers or fiducials.

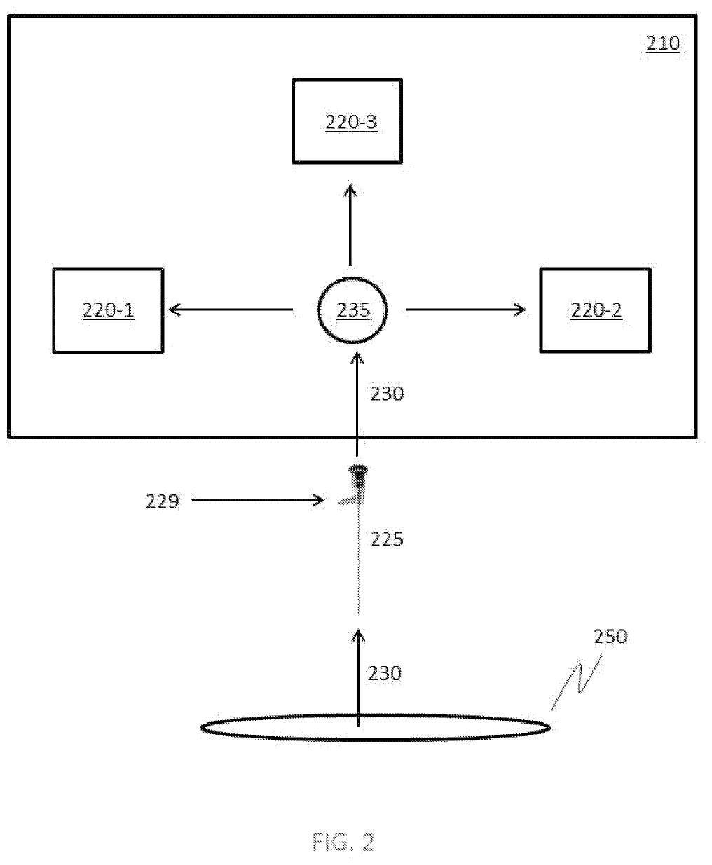

FIG. 2 illustrates an example imaging system that can be used compatibly with the light sources and imaging sensors described herein. The light sources may be used to generate one or more light signals for multispectral, hyperspectral, and/or mosaic imaging. The light signals may be transmitted to or through one or more portions of a surgical scene. The light signals may be reflected back and/or transmitted to the one or more multispectral, hyperspectral, and/or mosaic imaging sensors described above.

In some embodiments, the imaging system may comprise an imaging module 210. The imaging module 210 may be operatively coupled to a scope 225. The scope 225 may be configured to receive one or more input light signals 229 from one or more light sources. The one or more input light signals 229 may be transmitted from the one or more light sources to the scope 225 via a light guide. The one or more input light signals 229 may comprise, for example, white light for RGB imaging, fluorescence excitation light for fluorescence imaging, infrared light for laser speckle imaging, and/or time of flight (TOF) light for depth imaging. In some cases, the one or more input light signals 229 may comprise a first set of light signals for fluorescence imaging and a second set of light signals for at least one of RGB imaging, laser speckle imaging, or depth imaging. In some cases, the input light signals 229 generated by the plurality of light sources may comprise fluorescence excitation light having a wavelength that ranges from about 400 nanometers to at most about 500 nanometers, laser speckle light having a wavelength that ranges from about 800 nanometers to about 900 nanometers, and/or TOF light having a wavelength that ranges from about 800 nanometers to about 900 nanometers. In some cases, the fluorescence excitation light may have a wavelength of about 470 nanometers. In some cases, the laser speckle light may have a wavelength of about 852 nanometers. In some cases, the TOF light may have a wavelength of about 808 nanometers. The one or more input light signals 229 may be transmitted through a portion of the scope 225 (e.g., as a combined light beam or a series of light pulses) and directed to a target region 250.

In some cases, at least a portion of light signals transmitted to the target region 250 may cause a feature, a marker, or a biological material or tissue in the target region 250 to fluoresce. One or more fluorescence signals 230 may be produced by the feature, marker, or biological material or tissue in the target region 250 in response to the transmitted light signals. The one or more fluorescence signals 230 may be received at the imaging module 210 via the scope 225.

In some cases, a third set of light signals may be received at the imaging module 210 from the target region 250. The third set of light signals may correspond to the first set of light signals and/or the second set of light signals transmitted to the surgical scene 250 using the one or more light sources. The third set of light signals may comprise any optical signals that are reflected, emitted, or received from the surgical scene. In any of the embodiments described herein, the third set of light signals may comprise the one or more fluorescence signals 230 emitted from the target region 250.

In some embodiments, the imaging module 210 may be configured to receive the third set of light signals and direct the third set of light signals to one or more mosaic sensors. In some cases, the one or more mosaic sensors may be configured to perform multispectral, hyperspectral, and/or mosaic imaging of the surgical scene based on the third set of light signals.

In other embodiments, the imaging module 210 may be configured to receive the third set of light signals, and to direct different subsets or portions of the received light signals to different imaging sensors (e.g., imaging sensors 220-1, 220-2, 220-3) to enable various types of imaging based on different imaging modalities. The various types of imaging may be performed simultaneously and in real-time as the third set of light signals are being received. In some cases, the one or more imaging sensors may be releasably coupled to the imaging module 210. In other cases, the one or more imaging sensors 220-1, 220-2, 220-3 may be integrated with the imaging module 210 or a housing or other structural component or subcomponent of the imaging module 210.

In some embodiments, the imaging module 210 may be capable of performing multispectral, hyperspectral, and/or mosaic imaging of the surgical scene without requiring an optical element (e.g., a beam splitter or a dichroic mirror) to separate the received light signals by wavelength or direct wavelength-specific light to certain imaging sensors specifically configured for imaging based on predetermined wavelengths or spectral ranges. In some cases, the mosaic sensors described herein can be used to perform multi-wavelength imaging across a plurality of different spectral ranges. Such multi-wavelength imaging may correspond to various different types of imaging modalities, including, for instance, fluorescence imaging, laser speckle imaging, visible light or RGB imaging, and/or depth imaging.

In other embodiments, the imaging module 210 may comprise one or more optical elements 235 for splitting the third set of light signals into the different subsets of light signals. Such splitting may occur based on a wavelength of the light signals, or a range of wavelengths associated with the light signals. The optical elements 235 may comprise, for example, a mirror, a lens, or a prism. The optical elements 235 may comprise a dichroic mirror, a trichroic mirror, a dichroic lens, a trichroic lens, a dichroic prism, and/or a trichroic prism. In some cases, the optical elements 235 may comprise a beam splitter, a prism, or a mirror. In some cases, the prism may comprise a trichroic prism assembly. In some cases, the mirror may comprise a fast steering mirror. In some cases, one or more optical elements 235 may be placed adjacent to and/or in optical communication with each other to enable selective splitting and redirection of light signals to multiple different imaging devices or imaging sensors based on one or more properties of the light signals (e.g., wavelength, frequency, phase, intensity, etc.).

The third set of light signals received from the target region 250 may be directed through the scope 225 to the one or more optical elements 235 in the imaging module 210. In some cases, the one or more optical elements 235 may be configured to direct a first subset of the third set of light signals to an imaging sensor 220-1 for fluorescence imaging. In some cases, the one or more optical elements 235 may be configured to direct a second subset of the third set of light signals to an imaging sensor 220-2 for laser speckle imaging and/or TOF imaging. The first and second subsets of the third set of light signals may be separated based on a threshold wavelength. In some cases, the one or more optical elements 235 may be configured to permit a third subset of the third set of light signals to pass through to another imaging sensor 220-3. The imaging sensor 220-3 may comprise, for example, a camera for RGB imaging. In some cases, the imaging sensor 220-3 may comprise a third party camera that can be coupled to the imaging module 210. In some embodiments, the imaging module 210 may comprise a filter for the first imaging sensor 220-1. The filter may comprise, for example, a notch filter. The filter may be configured to block the fluorescence excitation light used to induce autofluorescence so that the imaging sensor 220-1 only sees the fluorescence signals produced by the target feature, marker, or biological material or tissue in the target region 250.

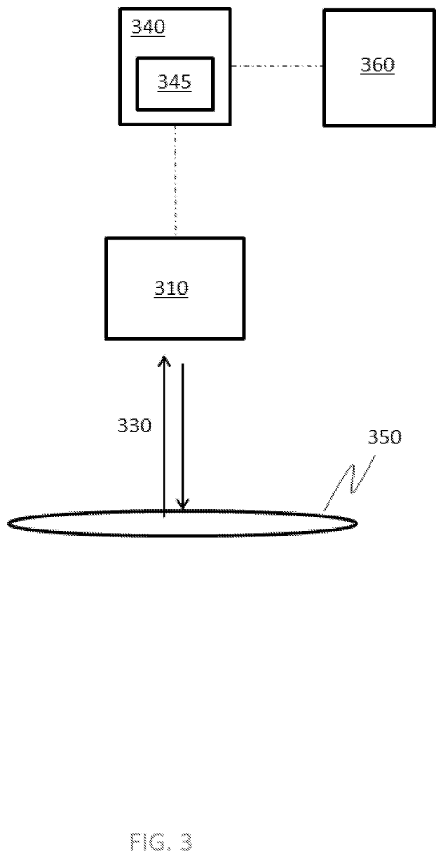

FIG. 3 illustrates an example of an imaging system comprising an imaging module 310, an image processing unit 340, a calibration module 345, and a display unit 360. The imaging module 310 may be configured to receive one or more optical signals 330 from a surgical scene 350. The optical signals may be registered using any of the multispectral, hyperspectral, and/or mosaic imaging sensors disclosed herein. In some cases, the light signals registered by the imaging sensors of the imaging module 310 may be processed by the image processing unit 340 to generate one or more medical images of the surgical scene 350. In some cases, the one or more medical images may comprise one or more multispectral, hyperspectral, and/or mosaic images.

As described elsewhere herein, in some cases, the imaging system may comprise a calibration module 345. The calibration module 345 may be configured to perform a calibration that correlates an amount of fluorescent light detected by the one or more imaging sensors to an amount or a concentration of fluorescent material present in one or more reference regions. The amount or the concentration of fluorescent material present in the one or more reference regions may be known or estimated previously using another imaging sensor or another imaging data set derived using the same imaging sensor. Once the calibration is completed, the imaging sensors may be used to image one or more select regions of a surgical scene. The amount of fluorescent light detected by the calibrated imaging sensors may be used to determine an amount or a concentration of fluorescent material present in the one or more select regions. The one or more select regions may be different than the one or more reference regions used for calibration purposes.

In some embodiments, the calibration module 345 may be configured to calibrate one or more light sources used to generate the light signals for imaging of the target site. For example, the calibration module 345 may be configured to adjust the wavelength or frequency of light emitted by the one or more light sources to the surgical scene, or a pulse timing of the one or more light sources.

In some embodiments, the calibration module 345 may be configured to calibrate the one or more imaging sensors. In some cases, the calibration module 345 may be configured to perform an intrinsic calibration. Intrinsic calibration may comprise adjusting one or more intrinsic parameters associated with the one or more imaging sensors. The one or more intrinsic parameters may comprise, for example, a focal length, principal points, a distortion, and/or a field of view. In some cases, the calibration module 345 may be configured to perform acquisition parameter calibration. Acquisition parameter calibration may comprise adjusting one or more operational parameters associated with image capture using the one or more imaging sensors. The one or more operational parameters may comprise, for example, a shutter width, an exposure, a gain, and/or a shutter timing.

In some cases, the calibration module 345 may be configured to calibrate the imaging system by sampling multiple targets at multiple illumination wavelengths or intensities. Such calibration may enhance the multispectral, hyperspectral, and/or mosaic imaging capabilities of the presently disclosed imaging systems by permitting fine tuning or adjustment of the operation of the light sources and/or the imaging sensors used for multispectral, hyperspectral, and/or mosaic imaging. The calibration may be performed automatically by a computer or manually by a user or an operator of the imaging system.

In some embodiments, the image processing unit 340 may be operatively coupled to a display unit 360. The display unit 360 may comprise a screen or a display that is viewable by a doctor or a surgeon. The display unit 360 may be configured to provide a visualization of the surgical scene 350 based on the one or more multispectral, hyperspectral, and/or mosaic images. In some cases, the visualization of the surgical scene 350 may comprise a dynamic overlay of images that is adjustable by the doctor or the surgeon depending on the operator preference or the needs of the current surgical procedure.

### Overlays

In some cases, the image processing module may be configured to generate one or more image overlays comprising the one or images generated using the image processing module. The one or more image overlays may comprise a superposition of at least a portion of a first image on at least a portion of a second image. The first image and the second image may be associated with different imaging modalities (e.g., fluorescence imaging, TOF imaging, laser speckle imaging, RGB imaging, etc.). The first image and the second image may correspond to a same or similar region or set of features of the surgical scene. Alternatively, the first image and the second image may correspond to different regions or sets of features of the surgical scene. The one or more images generated using the image processing module may comprise the first image and the second image.

In some cases, the image processing module may be configured to provide or generate an overlay of a perfusion map and a live image of a surgical scene. In some cases, the image processing module may be configured to provide or generate an overlay of a perfusion map and a pre-operative image of a surgical scene. In some cases, the image processing module may be configured to provide or generate an overlay of a pre-operative image of a surgical scene and a live image of the surgical scene, or an overlay of a live image of the surgical scene with a pre-operative image of the surgical scene. The overlay may be provided in real time as the live image of the surgical scene is being obtained during a live surgical procedure. In some cases, the overlay may comprise two or more live images or videos of the surgical scene. The two or more live images or videos may be obtained or captured using different imaging modalities (e.g., fluorescence imaging, TOF imaging, RGB imaging, laser speckle imaging, etc.).

In some cases, the image processing module may be configured to provide augmented visualization by way of image or video overlays, or additional video data corresponding to different imaging modalities. An operator using the imaging systems and methods disclosed herein may select various types of imaging modalities or video overlays for viewing. In some examples, the imaging modalities may comprise, for example, tissue autofluorescence imaging, ICG fluorescence imaging, RGB imaging, laser speckle imaging, time of flight depth imaging, or any other type of imaging using a predetermined range of wavelengths. The video overlays may comprise, in some cases, perfusion views and/or tissue autofluorescence views. Such video overlays may be performed in real-time. The overlays may be performed live when a user toggles the overlay using one or more physical or graphical controls (e.g., buttons or toggles). The various types of imaging modalities and the corresponding visual overlays may be toggled on and off by the user as desired (e.g., by clicking a button or a toggle). In some cases, the image processing module may be configured to provide or generate a first processed image or video corresponding to a first imaging modality (e.g., tissue autofluorescence) and a second processed video corresponding to a second imaging modality (e.g., laser speckle, TOF, RGB, etc.). The user may view the first processed video for a first portion of the surgical procedure, and switch or toggle to the second processed video for a second portion of the surgical procedure. Alternatively, the user may view an overlay comprising the first processed video and the second processed video, wherein the first and second processed video correspond to a same or similar time frame during which one or more steps of a surgical procedure are being performed.

In some cases, the image processing module may be configured to process or pre-process medical imaging data (e.g., surgical images or surgical videos) in real-time as the medical imaging data is being captured. Such processing or post-processing may comprise, for example, image alignment for a plurality of images obtained using different types of imaging modalities.

In some embodiments, the system may comprise a processing unit configured to (i) identify one or more critical structures in or near the surgical scene or (ii) distinguish between different critical structures in or near the surgical scene, based at least in part on the one or more images captured using the one or more imaging devices. The one or more critical structures may comprise, for example, a ureter, a bile duct, one or more blood vessels, an artery, a vein, one or more nerves, or one or more lymph nodes.

### Quantitative Applications

The light collected using the imaging sensors of the present disclosure may be used to generate, for example, a quantitative map of fluorescence indicating how much light is being emitted from each region of the surgical scene on a pixel-by-pixel basis. The quantitative applications described herein may provide several advantages over other existing imaging systems and methods. For instance, the quantitative map of fluorescence may show numerically how much fluorescent material is actually present in a region, instead of just a relative brightness to visually indicate that a fluorescent material or feature has been detected. This can allow a doctor or a surgeon to identify and interpret minute differences in the concentrations of a fluorescent material across different regions within an image, or across different images altogether. Such a feature is greatly advantageous over other imaging systems that can only provide a visual approximation of the concentration of a fluorescent material in an image, which visual approximation can be inconsistent for different doctors or surgeons. The systems and methods described herein may enable accurate interpretation and analysis of images in which two regions have a same concentration of fluorophores but the fluorescence from both regions are visually different.

In some cases, the imaging applications described herein may be enhanced using one or more calibration procedures. For example, one or more calibration procedures may be performed to correlate an amount of light emitted from a reference region to a concentration of fluorophores or an amount of fluorescent material in the reference region. In some cases, one or more calibration procedures may be performed to enhance interpretation and analysis of images in which two regions have a same concentration of fluorophores but the fluorescence from both regions are visually different.

In some cases, the imaging applications described herein may be enhanced by estimating an amount of blood in at least one of the two regions and compensating for the apparent differences in visual fluorescence based on the estimated concentration of blood. As described elsewhere herein, the concentration of blood may be estimated using one or more of the multispectral, hyperspectral, or mosaic imaging sensors presently disclosed. Such compensation may inform and enable the quantitative fluorescence applications described elsewhere herein. In some cases, the compensation may involve or enable a post-processing adjustment of one or more fluorescence images of a surgical scene to modify or scale the relative fluorescence from the two different regions to provide an enhanced fluorescence image that can provide doctors or surgeons with a more visually descriptive view of the surgical scene that can be easily interpreted to understand the relative fluorescence characteristics of one or more regions of the surgical scene relative to other regions in or near the surgical scene.

### Image Processing Module

In some embodiments, the system may further comprise an image processing module operatively coupled to any of the imaging sensors described herein. The image processing module may be configured to generate one or more enhanced or processed images of the surgical scene based on various light signals obtained or registered using the sensors described herein.

In some embodiments, the system may further comprise an image processing unit configured to generate one or more images of the surgical scene based on a set of light signals obtained or registered using the sensors described herein. The one or more images may comprise a first set of images and a second set of images of the surgical scene. In some embodiments, the image processing unit may be configured to adjust, modify, correct, or update the first set of images based on the second set of images. The first set of images and the second set of images may be obtained using a same sensor or different sensors associated with different types of imaging modalities. In some embodiments, the image processing unit may be configured to overlay at least one image from the first set of images on at least one image from the second set of images. In some embodiments, the image processing unit may be configured to overlay at least one image from the second set of images on at least one image from the first set of images.

In some cases, the image processing module may be configured to utilize image interpolation to account for a plurality of different frame rates and exposure times associated with the one or more imaging sensors when generating the one or more images of the surgical scene. In some cases, the image processing module may be configured to quantify or visualize perfusion of a biological fluid in, near, or through the surgical scene based on the one or more images of the surgical scene. In some cases, the image processing module may be configured to generate one or more perfusion maps for one or more biological fluids in or near the surgical scene, based on the one or more images of the surgical scene. In some cases, the image processing module may be configured to update, refine, or normalize the one or more perfusion maps based on an inferred hemoglobin density that is derived from one or more fluorescence measurements or fluorescent signals obtained using autofluorescence imaging. In some cases, the image processing module may be configured to update, refine, or normalize the one or more perfusion maps based on a distance between (i) a scope through which the plurality of light signals are transmitted and (ii) one or more pixels of the one or more images. In some cases, the image processing module may be configured to update, refine, or normalize the one or more perfusion maps based on a position, an orientation, or a pose of a scope through which the plurality of light signals are transmitted relative to one or more pixels of the one or more images. In some cases, the image processing module may be configured to update, refine, or normalize the one or more perfusion maps based on depth information or a depth map associated with the surgical scene. In some cases, the image processing module may be configured to determine a pose of a scope through which the plurality of light signals are transmitted relative to one or more pixels of the one or more images, based on depth information or a depth map. In some cases, the image processing module may be configured to update, refine, or normalize one or more velocity signals associated with the perfusion map based on the pose of the scope relative to the surgical scene. In some cases, the image processing module may be configured to update, refine, or normalize the one or more perfusion maps based on a type of tissue detected or identified within the surgical scene. In some cases, the image processing module may be configured to update, refine, or normalize the one or more perfusion maps based on an intensity of at least one of the first and second set of light signals. The intensity of the light signals may be a function of a distance between a scope through which the plurality of light signals are transmitted and one or more pixels in the surgical scene. In some cases, the image processing module may be configured to update, refine, or normalize the one or more perfusion maps based on a spatial variation of an intensity of at least one of the first and second set of light signals across the surgical scene. In some cases, the image processing module may be configured to infer a tissue type based on an intensity of one or more light signals reflected from the surgical scene, wherein the one or more reflected light signals comprise at least one of the first set of light signals and the second set of light signals. In some cases, the image processing module may be configured to use at least one of the first set of light signals and the second set of light signals to determine a time-varying motion of a biological material in or near the surgical scene.

In some cases, the imaging devices and/or the image processing module may be configured to (i) generate one or more fluorescence images based on the first set of light signals and/or the second set of light signals, and (ii) use the one or more fluorescence images to generate one or more machine-learning based inferences. The one or more machine-learning based inferences may comprise at least one of automatic video de-identification, image segmentation, automatic labeling of tissues or instruments in or near the surgical scene, and optimization of image data variability based on one or more normalized RGB or perfusion features. In some cases, the image processing module may be configured to (i) generate one or more fluorescence images based on at least one of the first set of light signals and the second set of light signals, and (ii) use the one or more fluorescence images to perform temporal tracking of perfusion and/or to implement speckle motion compensation.

In some cases, the image processing module may be operatively coupled to one or more 3D interfaces for viewing, assessing, or manipulating the one or more images. For example, the image processing module may be configured to provide the one or more images to the one or more 3D interfaces for viewing, assessing, or manipulating the one or more images. In some cases, the one or more 3D interfaces may comprise video goggles, a monitor, a light field display, or a projector.

In some cases, the image processing module may be configured to generate fluorescence images based at least in part on one or more fluorescence measurements obtained using the imaging devices or imaging sensors described herein. In some cases, the image processing module may be integrated with one or more imaging devices or imaging sensors. The fluorescence images may comprise an image or an image channel that contains information relating to fluorescence signals received or emitted from one or more surfaces or regions within the surgical scene. The fluorescence images may comprise fluorescence intensity or wavelength values for a plurality of points or locations within the surgical scene. The fluorescence intensity or wavelength values may be a function of and/or may correspond to a distance between (i) a tissue autofluorescence imaging sensor or a tissue autofluorescence imaging device and (ii) a plurality of points or locations within the surgical scene.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

### Computer Systems

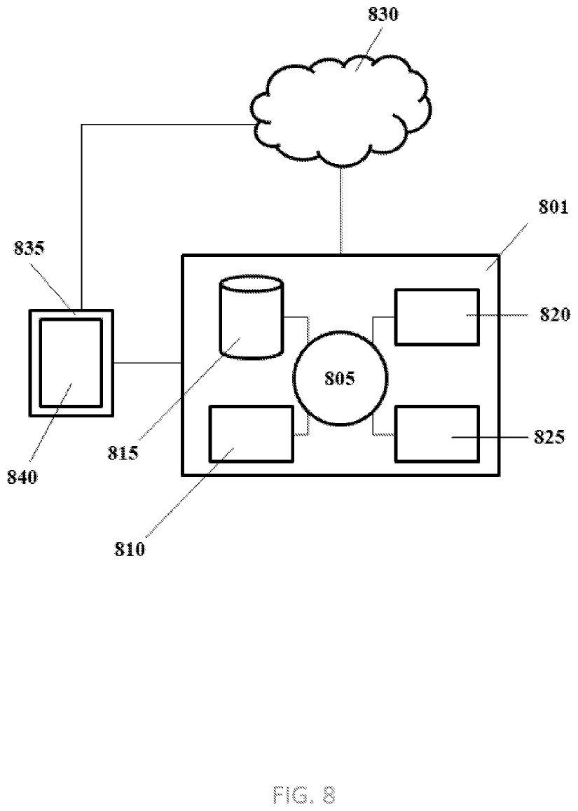

In another aspect, the present disclosure provides computer systems that are programmed or otherwise configured to implement methods of the disclosure. Referring to FIG. 8, the computer system 801 may be programmed or otherwise configured to implement a method for multispectral imaging. The computer system 801 may be configured to, for example, control a transmission of a plurality of light signals to a surgical scene. At least a portion of the plurality of light signals may interact with one or more features in the surgical scene, and one or more light signals may be emitted or reflected from the surgical scene. The one or more emitted or reflected light signals may be received at an imaging module. One or more optical elements of the imaging module may be used to direct a first subset of the emitted or reflected light signals to a first imaging unit and a second subset of the emitted or reflected light signals to a second imaging unit. The system may be further configured to generate one or more images of the surgical scene based on at least the first subset and second subset of emitted or reflected light signals respectively received at the first and second imaging units. The first subset and/or second subset of reflected light signals may be used for quantitative fluorescence imaging, RGB imaging, laser speckle imaging, time of flight (TOF) imaging, and/or any type of quantitative analysis of the surgical scene or any substance, materials, features, or processes within the surgical scene. The computer system 801 can be an electronic device of a user or a computer system that is remotely located with respect to the electronic device. The electronic device can be a mobile electronic device.

The computer system 801 may include a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are located external to the computer system 801 (e.g., on a remote server that is in communication with the computer system 801 through an intranet or the Internet).

The computer system 801 can communicate with one or more remote computer systems through the network 830. For instance, the computer system 801 can communicate with a remote computer system of a user (e.g., a doctor, a surgeon, an operator of a medical instrument, a medical imaging device, or a medical robot, etc.). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, a portal for a doctor or a surgeon to view one or more medical images obtained using one or more multispectral imaging sensors. The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. For example, the algorithm may be configured to generate one or more dynamic image overlays based on the one or more medical images generated using light signals that are reflected or emitted from the surgical scene and received at one or more of the multispectral imaging sensors described elsewhere herein. The one or more image overlays may comprise, for example, quantitative fluorescence imaging data associated with the surgical scene or one or more features (e.g., anatomical features or physiological characteristics), fiducials, or markers present or detectable within the surgical scene. In some cases, the image overlays may further comprise time of flight (TOF) imaging data, laser speckle imaging data, and/or RGB imaging data associated with the surgical scene or one or more features (e.g., anatomical features or physiological characteristics), fiducials, or markers present or detectable within the surgical scene.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A system comprising:
one or more imaging sensors configured to capture an image of a tissue, wherein each of the one or more imaging sensors includes a plurality of pixels, wherein at least one pixel of the plurality of pixels includes a first plurality of sub-pixels sensitive to a first band or wavelength of light and a second plurality of sub-pixels sensitive to a second band or wavelength of light, wherein the first band or wavelength of light is distinct from the second band or wavelength of light, wherein each of the first plurality of subpixels and the second plurality of sub-pixels are configured to generate image data having distinct image modalities, wherein a first image modality is a color image and a second image modality is fluorescence imaging or laser speckle imaging, and
a processing unit operatively coupled to the one or more imaging sensors, wherein the processing unit is configured to perform a quantitative analysis of one or more features or fiducials that are detectable within the image of the tissue based on one or more light signals obtained or registered using each of the first plurality of sub-pixels and the second plurality of sub-pixels.

2. The system of claim 1, wherein the system further comprises a first optical illumination in a third band or wavelength of light and a second optical illumination in a fourth band or wavelength of light.

3. The system of claim 2, wherein the first optical illumination is selected to generate data for the first plurality of sub-pixels sensitive to the first band or wavelength of light, and wherein the second optical illumination is selected to generate data for the second plurality of sub-pixels sensitive to the second band or wavelength of light.

4.-17. (canceled)

18. The system of claim 1, wherein the first band or wavelength of light and the second band or wavelength of light correspond to distinct bands or wavelengths of visible light, infrared light, or ultraviolet light.

19. The system of claim 1, wherein the first band or wavelength of light is within the infrared, and wherein the second band or wavelength of light is in the visible or the ultraviolet.

20.-22. (canceled)

23. The system of claim 1 further comprising one or more band pass filters for filtering out one or more bands or wavelengths of light emitted, reflected, or received from the tissue.

24.-32. (canceled)

33. A method of quantitative imaging using multispectral images, the method comprising:
providing an image of a tissue, wherein the image includes data from one or more imaging sensors, wherein each of the one or more imaging sensors includes a plurality of pixels, wherein at least one pixel of the plurality of pixels includes a first plurality of subpixels sensitive to a first band or wavelength of light and a second plurality of sub-pixels sensitive to a second band or wavelength of light, wherein the first band or wavelength of light is distinct from the second band or wavelength of light; and
at a processing unit operatively coupled to the one or more imaging sensors, (i) performing a quantitative analysis of one or more features or fiducials that are detectable within the image of the tissue based on one or more light signals obtained or registered using each of the first plurality of sub-pixels and the second plurality of sub-pixels and (ii) generating image data including distinct image modalities from each of the first plurality of sub-pixels and the second plurality of sub-pixels, wherein a first image modality is a color image and a second image modality is fluorescence imaging or laser speckle imaging.

34-36. (canceled)

37. The method of claim 33, further comprising, at the processing unit, collecting the one or more light signals from each of the first plurality of sub-pixels and the second plurality of sub-pixels substantially in parallel.

38. The method of claim 37, further comprising, at the processing unit, performing the quantitative analysis substantially in real time based on the one or more light signals collected substantially in parallel.

39. The method of claim 33, wherein the quantitative analysis comprises a quantification an amount of fluorescence emitted from the surgical scene or a concentration of a fluorescing material or substance of the one or more features or fiducials, and wherein the quantification is determined using spectral fitting or absorption spectroscopy.

40.-42. (canceled)

43. The method of claim 33, wherein the quantitative analysis comprises an identification or classification of one or more tissue regions in the tissue based on the one or more light signals.

44. The method of claim 33, wherein the quantitative analysis comprises a multispectral classification of one or more tissue regions in the tissue based on the one or more light signals having a plurality of different wavelengths.

45. (canceled)

46. The method of claim 33, wherein the quantitative analysis comprises a determination of the real-time blood oxygenation based on the one or more light signals.

47. The method of claims 33, wherein the quantitative analysis comprises a quantitative speckle analysis based on the one or more light signals

48.-67. (canceled)

68. A system comprising:
a processing unit including an image of a tissue, wherein the image includes data from one or more imaging sensors, wherein each of the one or more imaging sensors includes a plurality of pixels, wherein at least one pixel of the plurality of pixels includes a first plurality of sub-pixels sensitive to a first band or wavelength of light and a second plurality of sub-pixels sensitive to a second band or wavelength of light, wherein the first band or wavelength of light is distinct from the second band or wavelength of light,
wherein the processing unit is configured to perform a quantitative analysis of one or more features or fiducials that are detectable within the image of the tissue based on one or more light signals obtained or registered using each of the first plurality of sub-pixels and the second plurality of sub-pixels, and
wherein the processing unit is configured to (i) estimate an amount of blood in the tissue and (ii) determine an amount or a concentration of fluorophores or fluorescent material present in the tissue based on (a) the estimated amount of blood and (b) at least a subject of the one or more light signals. cm 69.-83. (canceled)

84. The system of claim 68, wherein the processing unit is configured to quantify an amount of fluorescence emitted from the tissue or an amount of fluorescent material present in the tissue based on a lighting condition of the image, wherein the lighting condition includes an illumination bias, an illumination profile, or an illumination gradient of the image.

85.-90. (canceled)

91. The system of claim 68, wherein the processing unit is configured to generate one or more combined images of the tissue based on image data or image signals derived from each of the first plurality of sub-pixels and the second plurality of sub-pixels.

92. The system of claim 91, wherein the processing unit is configured to generate a quantitative map of fluorescence in the tissue based on the image data or image signals.

93. The system of claim 92, wherein the quantitative map of fluorescence indicates an amount or concentration of fluorescence material present in one or more regions of the tissue

94. The system of claim 93, wherein the processing unit is configured to perform a calibration that correlates an amount of fluorescent light detected by the one or more imaging sensors to the amount or concentration of fluorescent material present in the one or more regions.

95.-99. (canceled)
